(19) United States
(12) Patent Application Publication
(10) Pub. No.: US 2026/0183375 A1
(43) Pub. Date: Jul. 2, 2026

(54) COMPOSITIONS, DEVICES, SYSTEMS AND METHODS RELATING TO VACCINATION AND STERILE PROTECTION AGAINST MALARIA

(71) Applicant: MalarVX Inc., Seattle, WA (US)

(72) Inventors: Marion Avril, Bainbridge Island, WA (US); Zachary Ward MacMillen, Seattle, WA (US)

(73) Assignee: MalarVX, Inc., Seattle, WA (US)

(21) Appl. No.: 18/858,825

(22) PCT Filed: Apr. 24, 2023
(86) PCT No.: PCT/US2023/019674

§ 371 Date: Oct. 22, 2024

Related U.S. Application Data
(60) Provisional application No. 63/333,878, filed on Apr. 22, 2022.

(51) Int. Cl. A61K 39/015; A61K 31/7105; A61K 39/00; A61P 33/06

(52) U.S. Cl. CPC A61K 39/015; A61K 31/7105; A61P 33/06; A61K 2039/53; A61K 2039/54; A61K 2039/545; A61K 2039/55555; A61K 2039/572; A61K 2039/575; A61K 2039/70

(57) ABSTRACT

Systems, compositions, devices, methods, etc., provide improved anti-malaria immunological responses comprising making, providing and administering vaccines comprising specific RNA molecules such as self-replicating replicon RNA (repRNA) encoding proteins from Plasmodium such as the P. yoelii (Py) CS protein (CSP), including in some embodiments substantially target proteins encoding target antigens, for example a whole or substantially whole CSP in the repRNA. The prime-and-trap intervals for the administration of the vaccine can comprise administration of only a single dose of a repRNA-Non-encapsulating oil-in-water emulsion nanocarriers (e.g., LION™) component followed by administration of as few as 3 or 2 doses, or even just a single dose, of the WO component (e.g., RAS or genetically attenuated WO) at 0 day (same day), or 1, 2, 3, 4, 5, 10, 14, 15 days or 28 days later.

### CROSS-REFERENCE TO RELATED APPLICATIONS

The present application claims the benefit of copending U.S. Provisional Patent Application Ser. No. 63/333,878, filed Apr. 22, 2022 (Apr. 22, 2023 being a Saturday), which application is incorporated herein by reference in its entirety.

### BACKGROUND

Malaria, caused by Plasmodium parasites, remains one of the most devastating infectious diseases worldwide despite control efforts that have lowered morbidity and mortality. Generally speaking, the only P. falciparum (Pf) vaccine candidates to show field efficacy are those targeting the asymptomatic pre-erythrocytic (PE) stages of infection. The subunit (SU) RTS, S/AS01 vaccine is the only known licensed malaria vaccine to date, but is only modestly effective against malaria clinical disease. Like RTS, S/AS01E, the SU R21 vaccine candidate targets the pre-erythrocyte sporozoite (spz) circumsporozoite (CS) protein and elicit high titer antibodies that have provided high levels of protection from disease, but do not induce liver resident memory (Trm) CD8⁺T cell that are potent mediators of the pre-erythrocytic immunity for long-term protection. In contrast, whole-organism (WO) vaccines such as radiation attenuated sporozoites (RAS) elicit both high titer antibodies and Trm, and have achieved high levels of sterilizing protection. However, they require multiple intravenous doses, extended and expensive supply chains and requirements such as cold transfer, which are major limitations for vaccine mass administration, for example in terms of production and compliance.

Thus, there has gone unmet a need for compositions, systems, methods, etc., for anti-malarial vaccines that are efficient over time and/or extended delivery distance (e.g., from manufacturing site to inoculation location), effective against the malaria vector, rapidly administered and/or otherwise improved over existing anti-malaria vaccines.

The present systems and methods, etc., provide solutions to one or more of these needs, and/or one or more other advantages.

### SUMMARY

The present systems, compositions, devices, methods, etc., provide improved anti-malaria immunological responses comprising making, providing and administering vaccines comprising specific RNA molecules such as self-amplifying replicon RNA (repRNA) encoding proteins from Plasmodium such as the P. yoelii (Py) CS protein (CSP), including in some embodiments substantially target proteins encoding target antigens, for example a whole or substantially whole CSP in the repRNA. The vaccines can comprise an advanced oil-in-water emulsion nanocarrier such as a Lipid InOrganic Nanoparticle (LION™) administered to a patient in conjunction with whole organism (WO) radiation attenuated sporozoites (RAS) and can be used in a ‘prime-and-trap’ heterologous vaccination strategy. The prime-and-trap intervals for the administration of the vaccine can comprise administration of only a single dose of the repRNA-Non-encapsulating oil-in-water emulsion nanocarriers (e.g., LION™) component followed by administration of as few as 3 or 2 doses, or even just a single dose, of the WO component (e.g., RAS or genetically attenuated WO) at 0 day (same day), or 1, 2, 3, 4, 5, 10, 14, 15 days or 28 days later.

Certain embodiments of the strategy herein were assessed in three different schedules with prime-and-trap intervals of 0, 5 or 14 days between administration of the doses. All immunization schedules induced higher anti-PyCS antibodies than RAS alone and showed boosting by the RAS trapping dose. Prime-and-trap vaccination also induced PyCS-specific liver resident memory CD8⁺T cells in the short interval immunization group, and all prime-and-trap regimens provided sterile protection against Py SPZ challenge. Surprisingly, even the combination of PyCS repRNA LION™ priming and same-day RAS trapping on a same-day prime-and-trap vaccination regimen achieved sterile protection in the P. yoelii mouse model of malaria.

In some embodiments herein, Applicants used full-length encoded CS protein expressed by infectious spz, which protein is advantageous for motility and hepatic cell invasion. CS is composed of an N-terminal region that binds heparin sulfate proteoglycans (RI), an immunodominant central repeat region of four-amino-acid (NANP) that are the target of neutralizing antibodies, and a GPI-anchored C-terminal region containing a thrombospondin-like domain (RII) and T cell epitopes.

In one embodiment, the prime-and-trap vaccine strategy is accelerated by combining SU and WO approaches with five-day or same-day delivery, as well as the use of multiple antigens to broaden and strengthen protection conferred. In one variant, a heterologous vaccine of multiple self-replicating repRNA antigens is used with an advanced lipid-inorganic nanoparticle (LION™) carrier for cytoplasmic delivery, followed by administration of WO radiation-attenuated Plasmodium sporozoites (spz). Different schedules of immunization providing sterile protection support this accelerated prime-and-trap regimen, which may induce both antibodies and liver-resident T cells. Varying schedules of immunization may be used to yield sterile and long-term protection. Also, multi-antigen immune responses may be used to broaden protection.

Turning to a discussion of some embodiments and aspects herein, “prime-and-trap” vaccination methods combine a “priming” dose of an antigen-encoding nucleic acid, followed by a heterologous “trapping” dose of WO spz that naturally home to the liver^{1,2}. The resulting liver Trm cells are positioned to respond quickly and efficiently to liver stage parasites to achieve sterile protection.

Oil-in-water emulsion nanocarrier are a particularly effective means of delivering next-generation recombinant ribonucleic acid (RNA) vaccines. Suitable carriers can be nanoparticle carriers that can, for example, be solid or semi-solid nanoparticles. In some embodiments, the nanocarriers are emulsion-based delivery vehicles that utilize the framework of squalene-based adjuvants and modifies it with the addition of the cationic lipid DOTAP (1,2-dioleoyl-3-trimethylammonium-propane, chloride salt), for binding of the RNA to the nanoparticle surfaces. An example of a suitable nanocarrier is a Lipid InOrganic Nanoparticle (LION™).

Despite the numerous challenges to creating an effective anti-malaria vaccines, Applicants have developed a prime-and-trap, repRNA-non-encapsulating oil-in-water emulsion nanocarrier vaccine, including for example a nanocarrier/repRNA (e.g., LION/repRNA) vaccine, comprising as little as two-doses that can be administered on the same-day. This can be administered, in one embodiment, via intramuscular (IM) priming with repRNA-encoding full-length CS of Plasmodium yoelii (repRNA-PyCS) formulated with a LION nanoparticle carrier or followed by a single dose of an intravenous injection of WO RAS (or genetically attenuated WO) vaccine as a trap. The “prime-and-trap” approaches herein can use as little as one dose of WO spz rather than multiple doses, simplifying the use of WO vaccines. This two-component, same-day regimen has been found to be effective in mammals and engaged/invoked both humoral and cellular arms of the immune system.

In some embodiments, the present systems, devices and methods, etc., herein provide methods of vaccinating a patient against malaria, comprising:
administering at least one priming dose that consists of a nanoparticle carrier in combination with nucleic acids encoding one or more antigenic Plasmodia proteins or protein fragments; and then
administering at least one trapping dose that consists of one or more antigenic Plasmodia proteins or protein fragments.

The nanoparticle carriers can be lipid inorganic nanoparticles (LIONs), solid nanoparticles or semi-solid nanoparticles. The Plasmodia can be taken from a group consisting of the human and zoonotic pathogens Plasmodium falciparum, Plasmodium vivax, Plasmodium malariae, Plasmodium ovale, and Plasmodium knowlesi and the nucleic acid can be a replicating RNA operably contained within a viral vector.

The vector can be a viral vector and can be for example a Venezuelan Equine Encephalitis Virus (VEEV) or an adenovirus.

The at least one trapping dose can comprise attenuated sporozoites from a group consisting essentially of RAS, GAP, Pb and CVac, and can comprise a replicating nucleic acid encoding one or more antigenic Plasmodia proteins or protein fragments that generates a T-cell response to the one or more antigenic Plasmodia proteins or protein fragments therein. The T-cell response includes liver-resident T cells.

The priming dose can be administered before and on the same day as the trapping dose, or the priming dose can be administered first followed by the trapping dose(s) from 12-120 hours later, from 121 hours to 28 days later or more than 28 days later,

In some aspects, the present systems, devices and methods, etc., provide anti-malarial vaccines comprising lipid inorganic nanoparticle nanocarriers coupled to replicon RNA (repRNA) operably connected to at least one immunological component of a plasmodium CS protein. The anti-malarial vaccine can comprise oil-in-water emulsion nanocarriers comprising repliconRNA (repRNA) operably connected to a substantially full-length plasmodium CS protein.

The anti-malarial vaccine can be a part of an anti-malarial vaccine system, the system further comprises a trapping component comprising attenuated whole organism Plasmodium sporozoites. The attenuated whole organism Plasmodium sporozoites can be radiation attenuated sporozoites (WO RAS) and can be genetically attenuated sporozoites.

In some aspects, the present systems, devices and methods, etc., provide methods of inducing an anti-malarial immunological response in a mammal, the method comprising:
a) administering to the mammal the anti-malarial vaccines herein;
b) then administering to the mammal the trapping components herein; and,
c) thereby inducing the anti-malarial immunological response in the mammal.

The methods further can comprise administering the trapping component within 14 days or less, within 5 days or less, or within 1 day of administering the anti-malarial vaccine, or on the same day of administering the anti-malarial vaccine.

The trapping component can be administered intravascularly (IV) and the anti-malarial vaccine can be administered intramuscularly (IM). The Lipid InOrganic Nanoparticle nanocarriers or the oil-in-water emulsion nanocarriers can be mixed with B) the repRNA less than about 60 minutes before the administration to the mammal, and the Lipid InOrganic Nanoparticle nanocarriers or the oil-in-water emulsion nanocarriersare can between mixed with B) the repRNA less than about 30 minutes before the administration to the mammal.

These and other aspects, features and embodiments are set forth within this application, including the following Detailed Description and attached drawings. Unless expressly stated otherwise, all embodiments, aspects, features, etc., can be mixed and matched, combined and permuted in any desired manner. In addition, various references are set forth herein, including in the Cross-Reference To Related Applications, that discuss certain systems, apparatus, methods and other information; all such references are incorporated herein by reference in their entirety and for all their teachings and disclosures, regardless of where the references may appear in this application. Such references are not necessarily prior art to the current application.

### BRIEF DESCRIPTION OF THE DRAWINGS

FIG. 1: The repRNA-PyCS was cloned into the replicon vector and combined with LION formulation prior mice injection.

FIG. 2. Preliminary study in mice. A) repRNA-PyCS SU was used to Prime mice (5 μg, day 0) followed by a WO Py RAS SPZ Trapping dose at day 14. Two doses Prime-Boost of repRNA-PfCS or repRNA-PyCS (5 μg) or a single dose of RAS (on day 14) were used as controls. B) The immunogenicity was measured as the anti-CS lgG titers by ELISA two weeks post-prime (013) and post-trap dose (029). C) Level of INFy produced by splenic T cells was evaluated by ELISPOT 4 weeks postprime dose. GeneGun-PyCSP mice were used as positive control (ggCSP) D) Control of the parasitemia and delay of the pre-blood patency followed a WT Py infection. E) Protection efficacy indicated by the percentage value post-challenge (protected/unprotected mice).

FIG. 3. Accelerated vaccine induces sterile protection. A) Mice were primed with 5 μg repRNA-PyCS 1-day (light blue), 5-day (green) or 28-days (purple) prior the Trapping dose of WO Py RAS SPZ. Two doses of Prime-Boost of repRNA-PyCS (orange, 5 μg, 28-day) and Prime-Trap control repRNA-PfCS+RAS (blue, 28-day) were used as controls. B) Control of the parasitemia following a challenge with WT Py infection 28 days post-Trap. C) Protection efficacy indicated as the percentage value post-challenge (protected/unprotected mice).

FIG. 4. lmmunogenicity of repRNA-LION-CS multi-antigen vaccine. A) Mice were primed and boosted 14 days apart with 5 μg of SU repRNA-based vaccine. B) Titer Antibodies were measured 2 weeks post-Boost via ELISA against the specific tandem repeat for PyCS, or PfCS, or PvCS. RepRNA-PyCS (orange circle), repRNA-PfCS (open circle) or repRNA-PvCS (black circle), was injected alone (grey area), or as a mixture of 2 or 3 antigens, injected together or separately in the right (R+) and left (L+) leg. An irrelevant repRNA-GFP as used as a control.

FIG. 5. Synergy and protective efficacy of combined antibodies in mice. A passive transfer of antibodies against either CS or UIS3 was nonprotective on their own (0%), while a combination of both prior challenge with IV injection of sporozoites showed a synergy effect taking sterile protection from 0 to 64%.

FIG. 6. Preliminary data on a 5-day vaccine longevity. A) Mice were primed with 5 μg of SU repRNA-PyCS (green) 5 days prior the Trapping dose of WO Py RAS. Two doses of Prime-Boost repRNA-PyCS (orange) and 2-dose Prime-Trap repRNAPfCS+RAS (blue) were used as controls. B) CS+tetramer liver resident COB T cells were assessed by flow cytometry analysis 4 weeks post-Trap dose. C) Control of the parasitemia and delay of the pre-blood patency followed a WT Py infection 2 months post-Trap. D. Protection efficacy is indicated by the percentage value post-challenge (protected/unprotected mice).

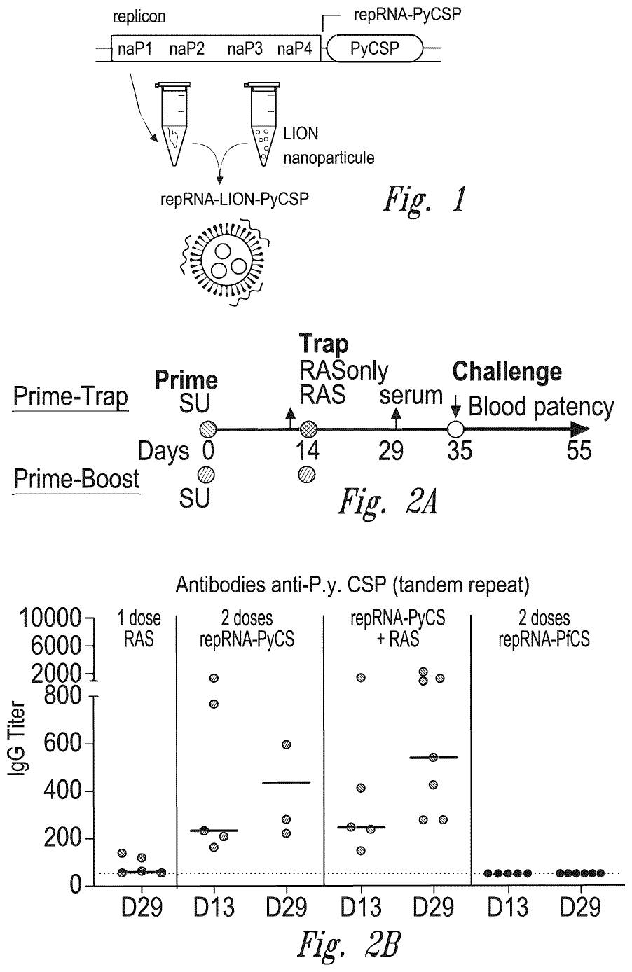

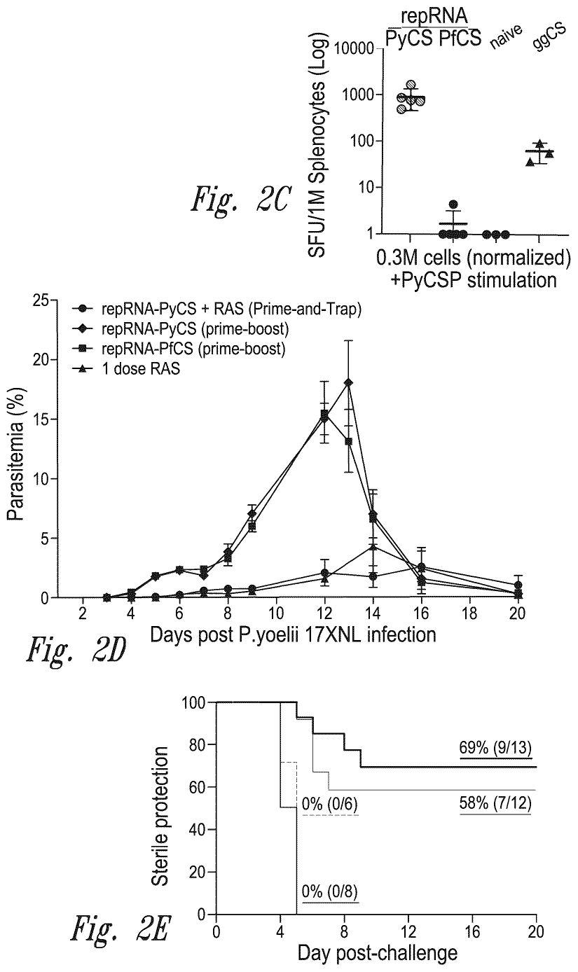

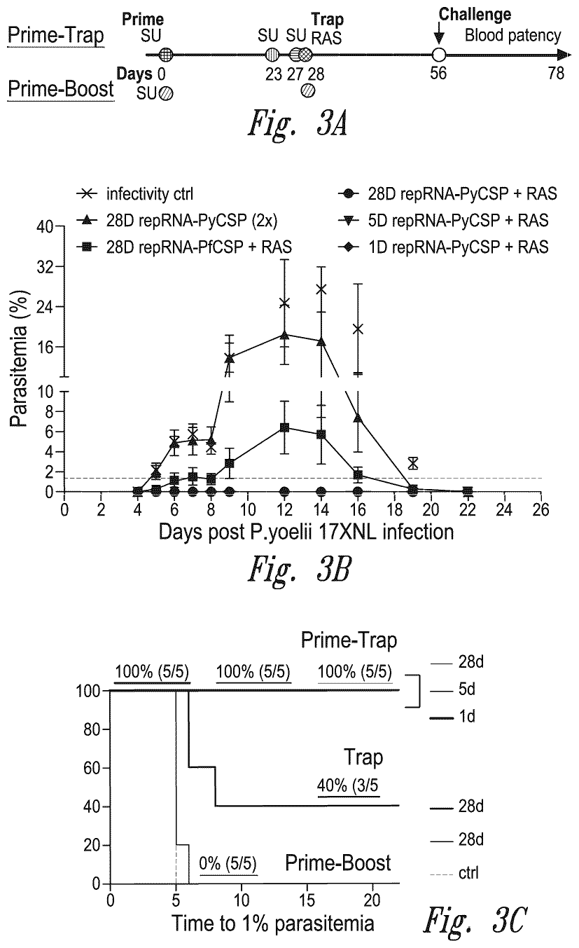

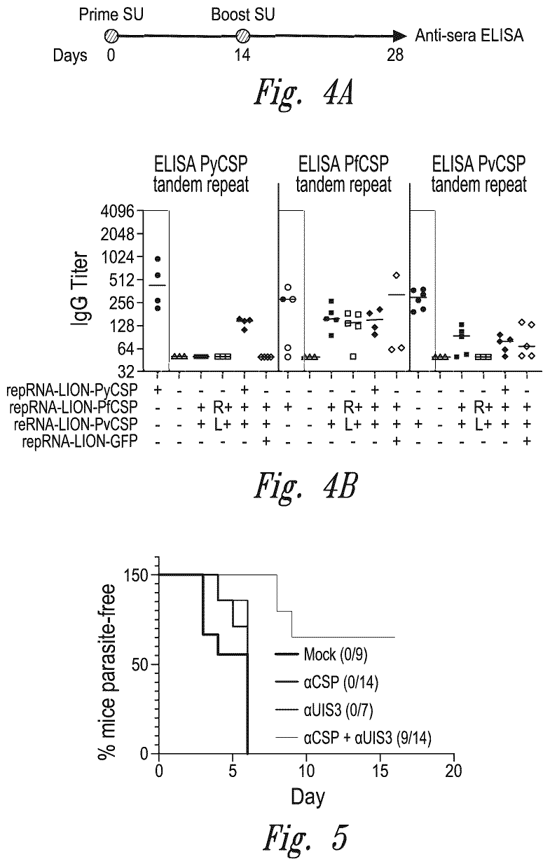

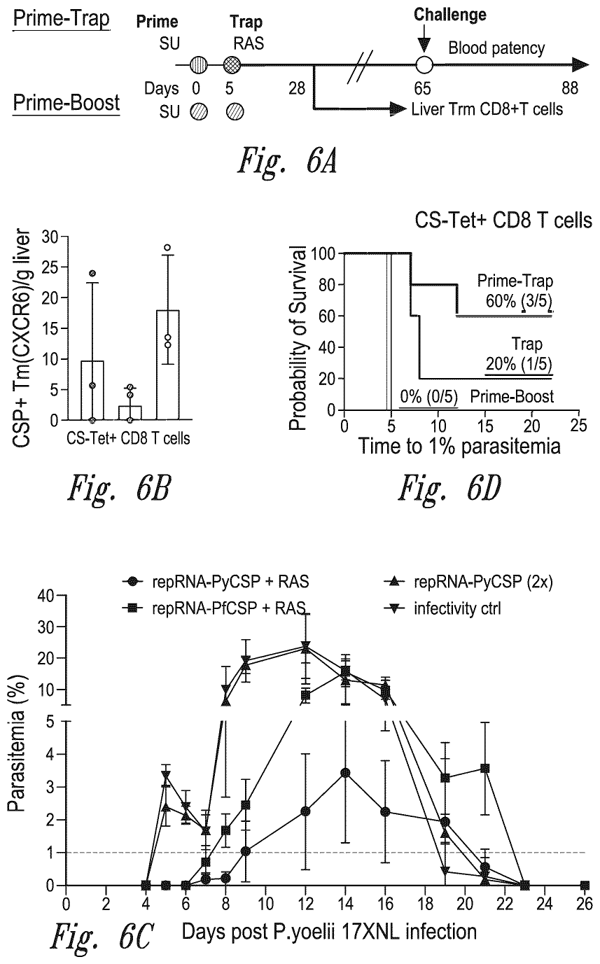

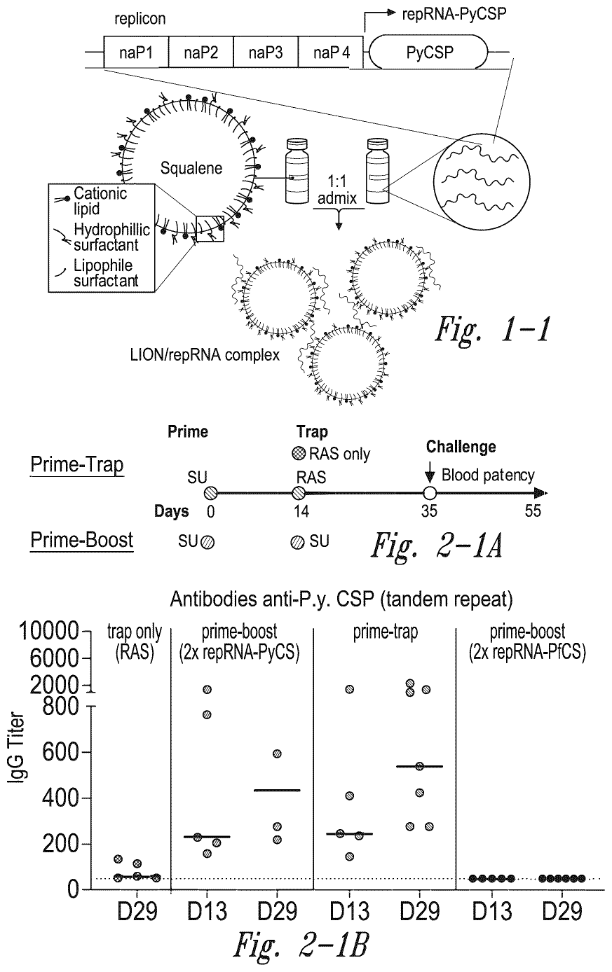

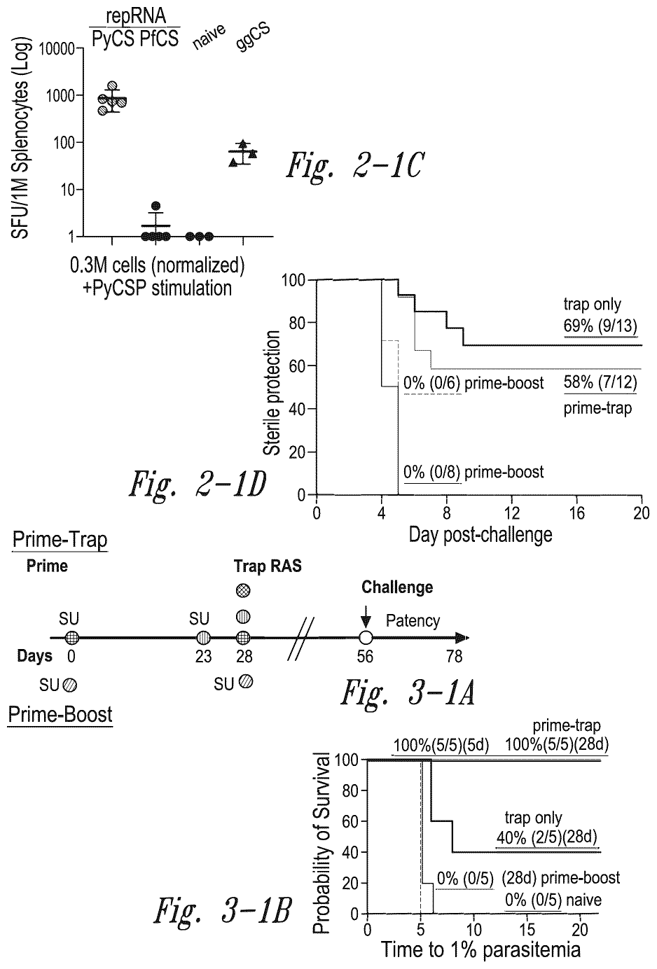

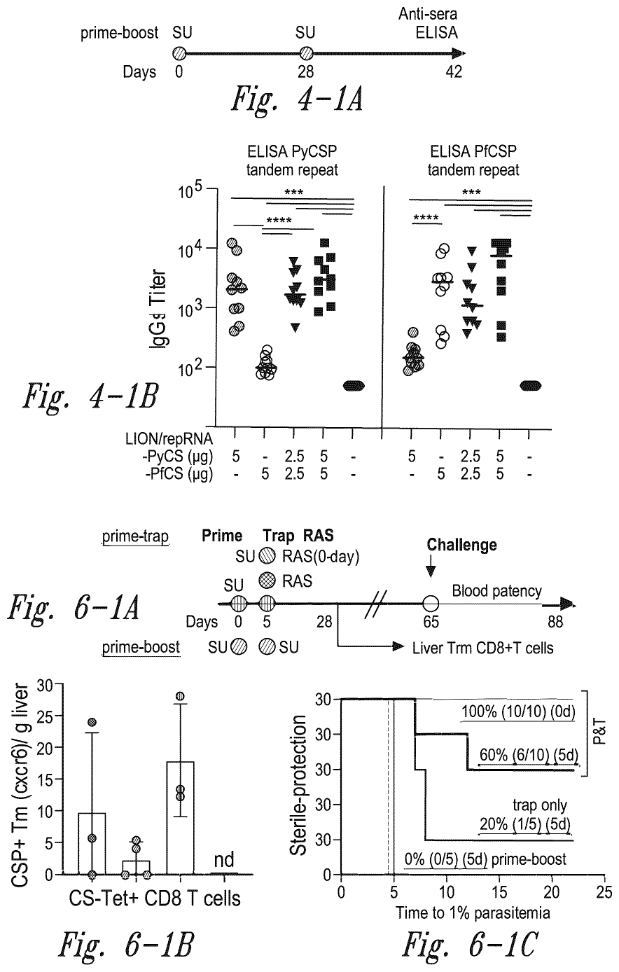

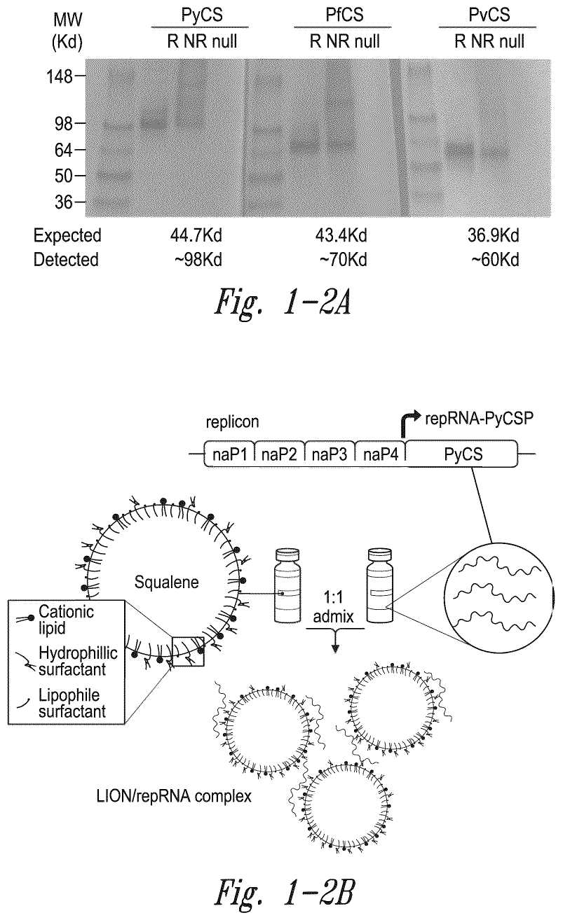

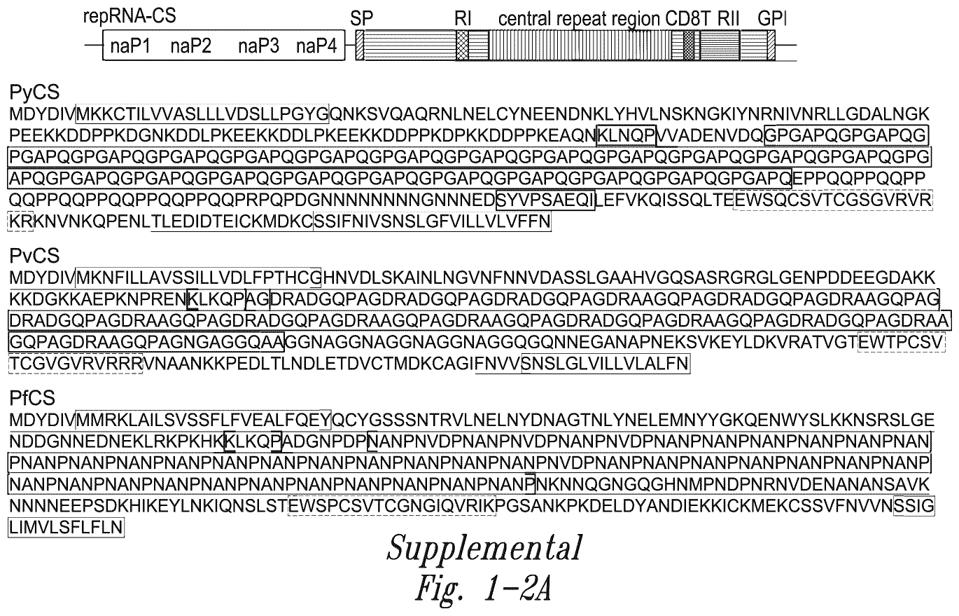

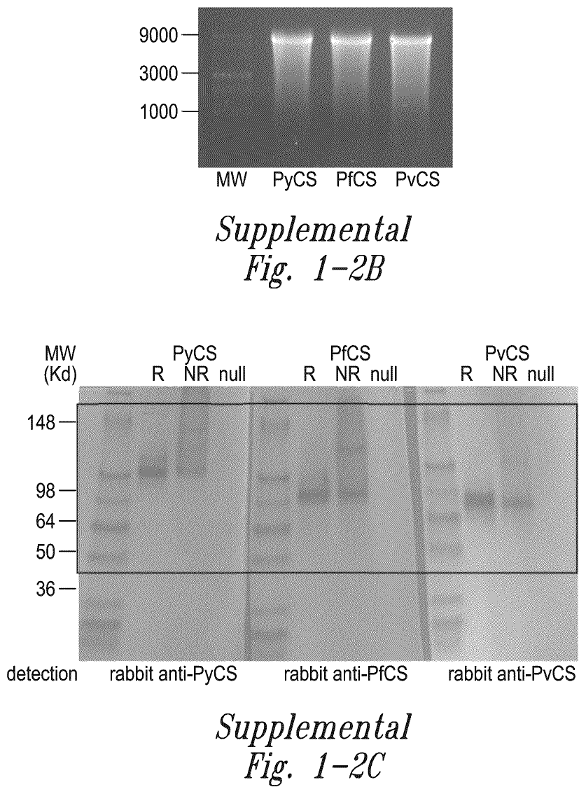

FIG. 1-1 shows RepRNA-PyCS/LION nanocarrier formulation prior to injection.

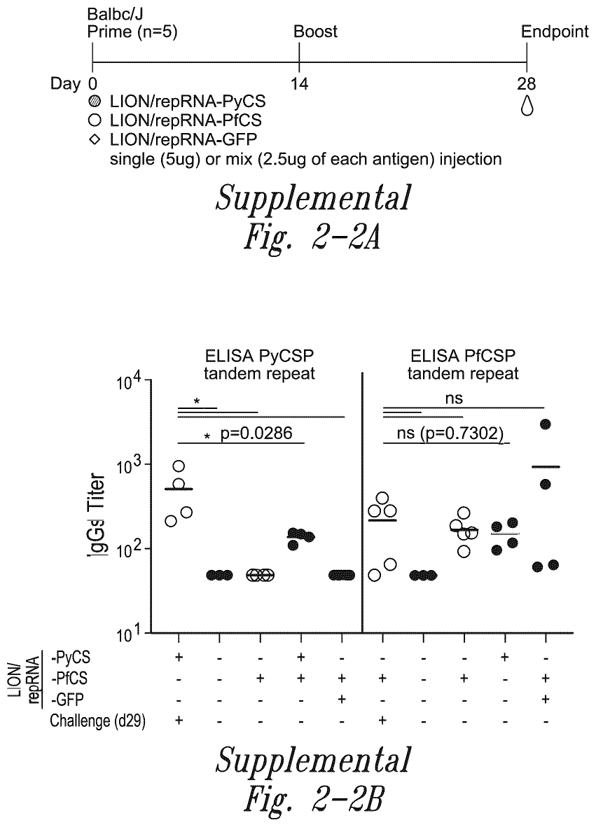

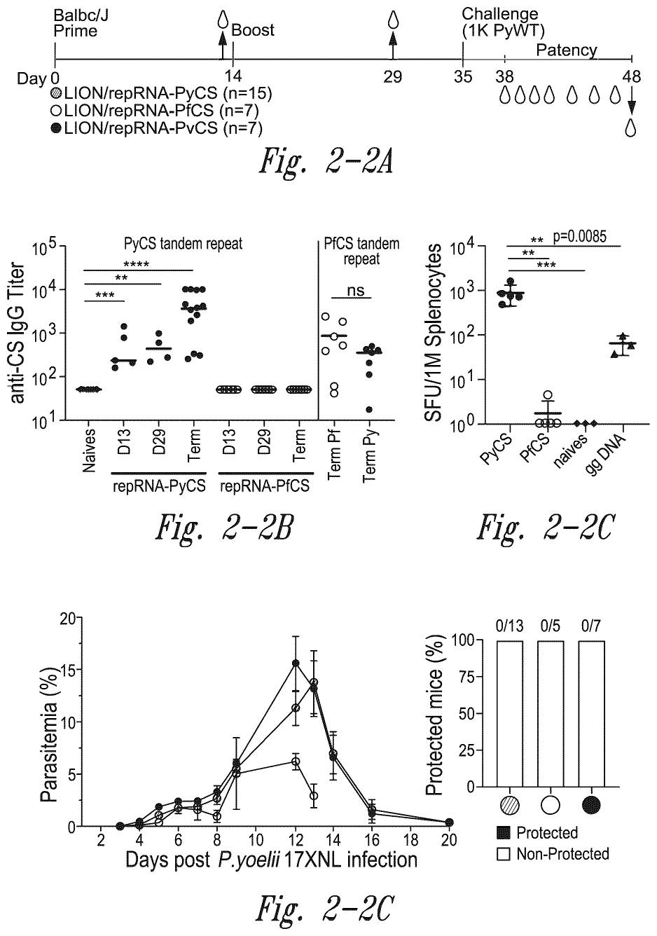

FIGS. 2-1A through 2-1D. lmmunogenicity studies in mice. A) repRNA-PyCS SU (5 μg) was used to prime mice followed byWO Py RAS SPZ trapping dose at day 14. Two doses (prime-boost) of repRNA-PfCS or repRNA-PyCS (5 μg) or single dose of RAS (on day 14) were used as controls. B) lmmunogenicity was measured as antics lgG titers by ELISA two weeks post-prime (D13) and post-trap dose (D29). C) Level of IFNy produced by splenic T cells was evaluated by ELISPOT 4 weeks post-prime dose. GeneGun-PyCSP mice were used as positive control (ggCSP). D) Protection efficacy defined as percentage of protected mice postchallenge (protected/total mice).

FIGS. 3-1A through 3-1B. Accelerated vaccine induces sterile i:1rotection. A) Mice were primed with 5 μg repRNA-PyCS, 5-day (green) or 28-day (purple) prior to trapping dose of WO Py RAS SPZ. Two doses (Prime-Boost) of repRNA-PyCS (orange, 5 μg, 28-day) and prime-trap control (trap only=repRNA-PfCS+RAS) (blue, 5 μg, 28-day) were used as controls. B) Protection efficacy defined as percentage of protected mice post-challenge (protected/total mice).

FIGS. 4-1A through 4-1B. lmmunogenicity of repRNA-LION-CS dual antigen vaccine. A) Mice were primed and boosted 28 days apart with repRNA-PyCS (orange circle), repRNAPfCS (open circle), both injected alone, or as mixture of 2×2.5 μg (triangle) and 2×5 μg (square) two repRNA. Naive mice sera were used as control. B) Titer lgGs were measured 4 weeks post-Boost via ELISA against specific PfCS or PyCS tandem repeat.

FIGS. 6-1A through 6-1C. Preliminary data on 5-day and 0-day vaccine durability. A) Mice were primed with 5 μg of SU repRNA-PyCS (green) 5 days or same-day (pink) prior to trapping dose of WO Py RAS. Two doses of prime-boost repRNA-PyCS (orange) and trap only repRNA-PfCS+RAS (blue) were used as controls. B) CS+tetramer liver resident cos+T cells were assessed by flow cytometry 4 weeks post-trap. C) Protection efficacy defined as percentage of protected mice post-challenge (protected/total mice).

FIG. 1-2A to 1-2B. LION/repRNA-CS vaccine design. After RNA transcription and capping, repRNA-PyCS or-PfCS or-PvCS was transfected into BHK cells and 24 to 48 hours later, the transfected cells lysate (R, reduced, NR, non-reduced) or null transfection used as control, were analyzed by Western blot, using the rabbit polyclonal antibodies for immunodetection. B) Graphic representation of replicon repRNA-PyCS and LION formulation that was used for mice immunization after mixing.

Supplemental FIGS. 1-2A to 1-2C. CS replicon and protein sequences. A) Schematic of the replicon encoding the full length of either P. yoelii, P. vivax or P. falciparum, circumsporozoite (CS) protein encoding including the signal peptide (SP) in N-terminal, the region I (RI), the entire central repeat region, the CD8⁺T cell epitope, the region II (RII) up to the GPI anchor signal in C-terminal, was cloned into an alphavirus replicon encoding the four nonstructural protein (nsP1 to nsP4) genes of the Venezuelan equine encephalitis virus (VEE) strain TC-83. For each CS cloned into the replicon, their full protein sequence is indicated. B) Denatured gel of repRNA-PyCS,-PfCS and PvCS cloned into the VEE vector. C) Uncropped FIG. 1B Western blot.

Supplemental FIGS. 2-2A to 2-2B. Immunogenicity of repRNA-CS formulated in LION in Balb/cJ. A) Mice immunization schedule for the 14-day Prime-Boost with either LION/repRNA-PyCS, or-PfCS. or LION/repRNA-GFP used as control replicon. n=5 mice per cohort in one experiment. B) Mice were either immunized with single antigen (5 μg each) or double antigens mixed together (2.5 μg each). Final bleeds were collected two weeks post-boost at endpoint and immune responses were analyzed by ELISA against their corresponding CS tandem repeat region. Each data point represents an individual mouse and the bar represents the group mean. Asterisk represents significance as determined by the non-parametric two-tailed Mann-Whitney U test (*p=0.05, **p=0.01, ***p=0.001, ****p<0.0001).

FIGS. 2-2A to 2-2C. Immunogenicity and efficacy of a prime-boost vaccine with repRNA encoding either PyCS (orange circle), PfCS (opened circle) or PvCS (black circle) formulated with LION. A) Balb/cJ mice immunization schedule. Mice were injected with 5 ug of LION/repRNA-PyCS, -PfCS, -PvCS in a 2-week interval prime-boost regimen. B) Mice sera were collected at post-prime (day 13), post-boost (day 29) and final bleeds post-challenge (day 48, ie. terminal bleed). PyCS and-PfCS antibody responses were determined by repeat region peptide PyCS or PfCS titration ELISA, respectively. C) Parasitemia and protection post-challenge of the three prime-boost cohorts. Number of mice per cohort indicated above bar graph. D) IFNg ELISPOT of CS-specific T cells four weeks after a single prime injection of LION/repRNA-PyCS. Gene gun DNA encoding PyCS (ggDNA), or LION/repRNA-PfCS or naïve cohorts were used as control. The n value represents total number of mice tested per cohort, in two to three independent assays. Each data point represents an individual mouse and the bar represents the group mean. Asterisk represents significance as determined by the non-parametric two-tailed Mann-Whitney two-tailed test (*p=0.05, **p=0.01, ***p=0.001, ****p<0.0001).

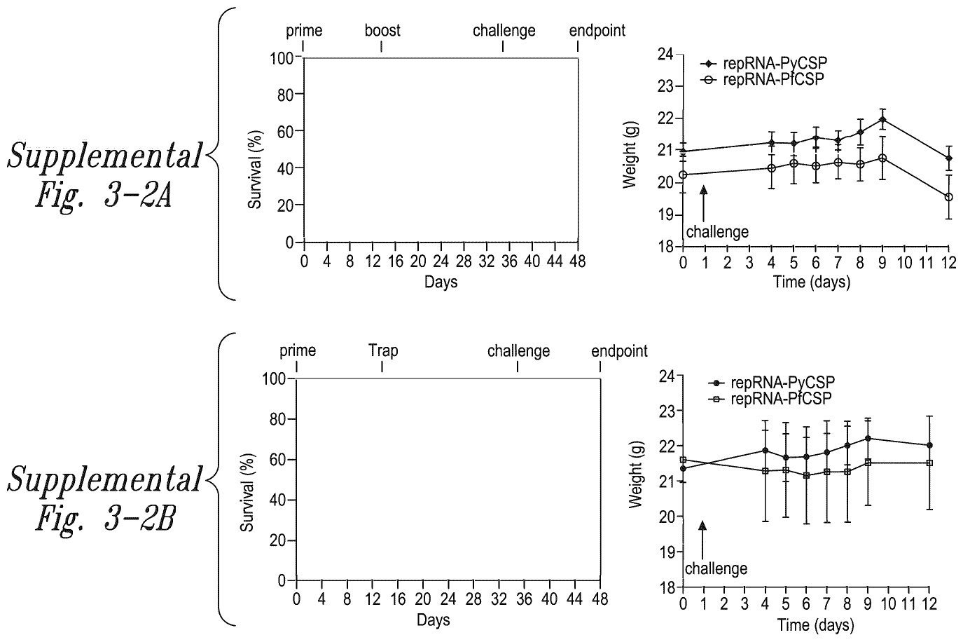

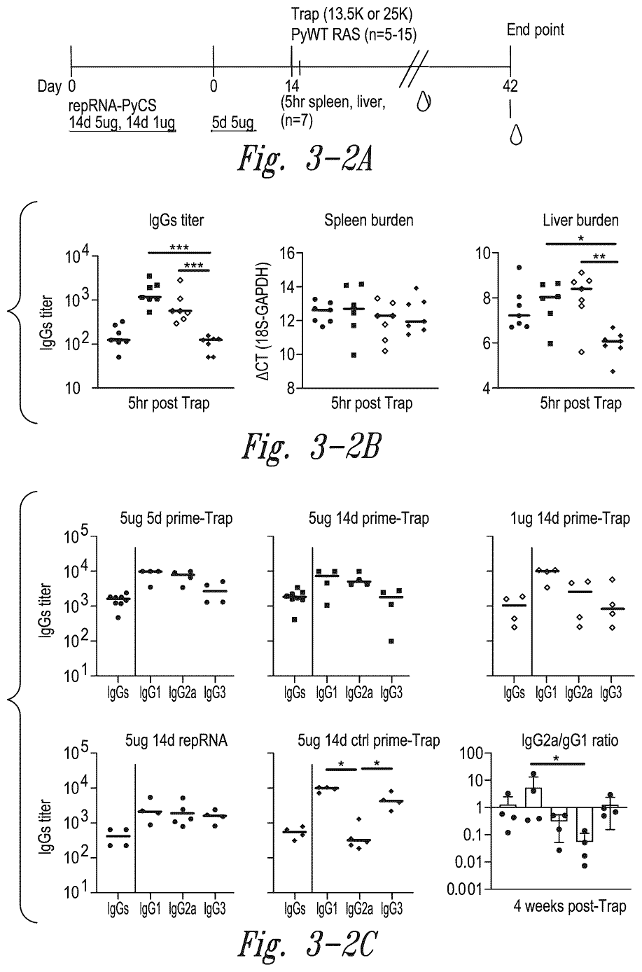

Supplemental FIGS. 3-2A to 3-2B. Survival and body weight curves of Balb/cJ immunized mice. Survival curves show live mice from the day of immunization (day 0) until endpoint of the study (day 48). Mice body weight was monitored after challenge with 17XNL spz given intravenously. Each point represents the mean of all mice per group (n=5 per cohort). A) Mice were immunized with prime-boost LION/repRNA-PyCS (orange curve) or LION/repRNA-PfCS (black curve). B) Mice were immunized with prime-and-trap LION/repRNA-PyCS +RAS (red curve) or single trap (blue curve) (n=5 per cohort).

FIGS. 3-2A to 3-2C. Immunogenicity of an accelerated prime-and-trap immunization regimens in Balb/cJ mice. A) 5 ug 5-day (green data) or 5 ug 14-day (red data) or 1 ug 14-day regimen of repRNA-PyCS (light blue data) prime followed by the trap dose of 25,000 RAS, were tested in mice. Control cohorts are prime-boost repRNA-PyCS (orange data) or trap cohort (repRNA-PfCS+RAS, dark blue data). A) Mice immunization schedule. B) Spleen, liver, and serum from seven mice per cohort were harvested 5 hours post-trap injection. Total IgGs titer was analyzed by ELISA, while spleen and liver parasite burden were analyzed by qPCR quantification. Spleen and liver burden qPCR was evaluated using One-way ANOVA followed by Kruskal-Wallis test and Dunn's multiple comparisons test (*p<0.05, **p<0.005). C) Final bleeds sera were collected at endpoint, 4 weeks post-trap (day 42) to evaluate the total IgGs titer and subclasses IgG1, IgG2a, IgG3 for each cohort by ELISA. Ratio of IgG2a/IgG1 is indicated in bar graph. All others statistical analyses were performed using a Mann-Whitney test. The n value represents total number of mice tested per cohort, in two independent assays. Each data point represents an individual mouse, and the bar represents the group mean.

FIGS. 4-2A To 4-2C. 5-day prime-and-trap and trap alone (RAS) regimens of immunization induce higher-frequency liver Trm cells than 14-day prime-and-trap regimen. A) Schedule of immunization. 5 ug 5-day (square, green data) or 5 ug 14-day (triangle, red data) or 1 ug 14-day regimen of repRNA-PyCS (inverted triangle, light blue data) prime followed by the trap dose of 25,000 RAS (dark blue data), were tested in mice. Control cohorts are prime-boost repRNA-PyCS (orange data) or trap cohort (repRNA-PfCS+RAS, black data). B) Flow cytometric total CD8⁺T cells and activated (CD44hi/CD62Llo) CD8⁺T cells in perfused livers 28 days after the Trap dose of 25,000 RAS. C) Flow cytometric analysis of tetramer-stained, CS-specific CD8⁺liver Trm cells (by CD69+ and either KLRG1lo (upper) or CXCR6+ (lower)). All error bars are SD of the mean. *p=0.05, **p=0.01, ***p=0.001 by Mann-Whitney two-tailed test. The n value represents total number of mice tested per cohort, in two independent assays. Each data point represents an individual mouse and the bar represents the group mean with error bars representing the standard error of the mean. Asterisk represents significance as determined by the non-parametric two-tailed Mann-Whitney U test (*p=0.05, **p=0.01, ***p=0.001, ****p<0.0001).

FIGS. 5-2A to 5-2E. Efficacy of an accelerated prime-and-trap immunization regimens in Balb/cJ mice. A) Mice immunization schedule. Prime dose of 5 ug 5-day (green data) or 5 ug 14-day (red data) or 1 ug 14-day regimen of repRNA-PyCS (light blue data) followed by the trap dose (dark blue data) of 25,000 RAS, were tested in mice. Three weeks later mice were challenged intravenously with 1,000 live spz isolated from infected mosquitos. Control cohorts are the trap cohort (RAS alone, dark blue data) and naïve mice cohort. Mice cohort showing partial protection were rechallenged with 1,000 live spz six weeks later. B) Protection post-challenge and re-challenge per cohort. Number of mice per cohort indicated above bar graph as protected/non-protected ratio. C) Parasitemia post-challenge of immunized mice cohorts. D) Sera from seven mice per cohort were harvested 20 days post-challenge (day 55) and CS specific IgGs titer were analyzed by ELISA. E) Final bleeds (six weeks post-rechallenge, day 104) of the four cohorts of mice that shows full protection. The n value represents total number of mice tested per cohort, in two or three independent assays. Each data point represents an individual mouse and the bar represents the group mean. All statistical analyses were performed using a Mann-Whitney two-tailed test.

FIGS. 6-2A to 6-2D. Prime-and-trap vaccine improves protection against stringent challenge in Balb/cJ mice. A) Schedule of immunization. prime-and-trap vaccine composed of a 5 ug 5-day regimen of prime with repRNA-PyCS (green data) followed by Trap dose of 25,000. Control cohort is a 5 ug 5-day prime-boost repRNA-PyCS (orange data) or a Trap cohort (repRNA-PfCS+RAS, dark blue data). Two months later mice were challenged intravenously with 10,000 live spz isolated from infected mosquitos. B) Parasitemia post-challenge of all cohorts including the naive mice cohort. Emphasized is the parasitemia peak at day 12, each dot representing a mouse, and the bar is the mean of the cohort. C) Patency curves of mice post-challenge per cohort. Number of mice per cohort indicated above bar graph as protected/non-protected ratio. ****p<0.0001 by Fisher exact test. D) Final bleeds sera were collected at endpoint, (day 87) to evaluate the total IgGs titer and subclasses IgG1, IgG2a, IgG3 for each cohort by ELISA. Ratio of IgG2a/IgG1 is indicated in bar graph. The n value represents total number of mice tested per cohort, in one experiment. Each data point represents an individual mouse and the bar represents the group mean. All statistical analyses were performed using a Mann-Whitney two-tailed test *p=0.05, **p=0.01, ***p=0.001 unless specified in panel.

FIGS. 7-2A to 7-2E. Prime-and-trap vaccine in C57BL/6 mice. A) Schedule of immunization. Prime-and-trap vaccine composed of a 5 ug 5-day regimen of prime with repRNA-PyCS (green data) followed by trap dose of 25,000. Control cohort is a 5 ug 5-day prime-Boost repRNA-PyCS (orange data) or a trap cohort (repRNA-PvCS+RAS, purple data) or double trap (RAS+RAS, dark blue data), and a cohort of naïve mice. One-month post-trap (day 35), sera was collected. Two months post-trap, mice were challenged intravenously with 5,000 live spz isolated from infected mosquitos. B) Post-trap sera were harvested (day 35) and CS specific IgGs titer were analyzed by ELISA. C) Patency curves (>1% parasitemia) of mice and protection post-challenge per cohort. Number of mice per cohort indicated above bar graph. D) Parasitemia post-challenge of all cohorts. Emphasized is the parasitemia peak at day 12, where each dot represents a mouse, and the bar is the mean of the cohort. E) Final bleeds sera were collected at endpoint, (day 87) to evaluate the total IgGs titer and subclasses IgG1, IgG2a, IgG3 for each cohort by ELISA. Ratio of IgG2a/IgG1 is indicated in bar graph. The n value represents total number of mice tested per cohort, in one experiment. Each data point represents an individual mouse and the bar represents the group mean. All statistical analyses were performed using a Mann Whitney two-tailed test *p=0.05, **p=0.01, ***p=0.001.

FIGS. 8-2A to 8-2G. Immunogenicity and efficacy of a same-day prime-and-trap vaccine in Balb/cJ mice. A) Schedule of immunization. Prime-and-trap vaccine composed of a 5 ug 5-day regimen (green data) or 5 ug same-day interval (pink data) of prime with repRNA-PyCS followed by trap dose of 25,000. Control cohort is 5 ug same-day interval of a trap cohort (repRNA-PfCS+RAS, dark blue data). Three weeks later mice were challenged intravenously with 1,000 live spz isolated from infected mosquitos. B) Parasitemia post-challenge of all cohorts including the naive mice cohort (black data). C) Patency curves (>1% parasitemia) of mice post challenge. D) Protection post-challenge per cohort. Number of protected mice per cohort indicated above bar graph. ****p<0.0001 by Fisher exact test. The n value represents total number of mice tested per cohort, in two independent experiments. E) Schedule of immunization of a same day prime-and-trap vaccine (5 ug, 0-day regimen) with Trap dose of 20,000 (cryopreserved spz) administered IM and IV the same day respectively. Control cohort is the naive mice cohort. Two months later mice were challenged intravenously with 20,000 cryopreserved spz isolated from infected mosquitos. F) Patency curves (>1% parasitemia) of mice post challenge. G) Patency curves (>1% parasitemia) and protection post-challenge per cohort. Number of protected mice per cohort indicated above bar graph. ****p<0.0001 by Fisher exact test. The n value represents total number of mice tested per cohort, in one experiment. All others statistical analyses were performed using a Mann Whitney two-tailed test *p=0.05, **p=0.01, ***p=0.001.

### DETAILED DESCRIPTION

present systems, compositions, devices, methods, etc., provide significantly improved, and even same-day administration, anti-malaria vaccines and immunization processes. Such methods, etc., include making, providing and administering vaccines comprising specific RNA molecules such as self-replicating RNA (repRNA) encoding proteins from Plasmodium such as the P. yoelii (Py) CS protein (CSP), including in some embodiments target proteins substantially encoding target antigens, for example a whole or substantially whole CSP in the repRNA. The vaccines can comprise an advanced oil-in-water emulsion nanocarrier such as a Lipid InOrganic Nanoparticle (LION™) administered to a patient in conjunction with whole organism (WO) radiation attenuated sporozoites (RAS) and can be used in a ‘prime-and-trap’ heterologous vaccination strategy. The prime-and-trap intervals for the administration of the vaccine can comprise administration of only a single dose of the repRNA-Non-encapsulating cationic nanocarriers (e.g., LION™) component followed by administration of as few as 3 or 2 doses, or even just a single dose, of the WO component (e.g., RAS or genetically attenuated WO) at 0 day (same day), or 1, 2, 3, 4, 5, 10, 14, 15 days or 28 days later.

### EXAMPLES

LION RNA nanoparticles are immunogenic in animals. When introduced into cells, repRNA initiates biosynthesis of antigen-encoding mRNA, raising and prolonging antigen expression and thereby enhancing humoral and cellular immune responses. Moreover, repRNA elicits more robust immune responses after a single dose than conventional mRNA formulations, offering an attractive approach for emerging infectious diseases, such as Dengue, Zika, and SARS-CoV-2. However, the in vivo instability of RNA and the requirement for transport through lipid bilayers have stimulated development of novel vehicles for intracellular RNA delivery. As another embodiment, alternative to LNP-encapsulated mRNA, Applicants have developed repRNA-CoV2S, a stable and highly immunogenic vaccine candidate comprising repRNA formulated with a Lipid InOrganic Nanoparticle (LION™) to enhance vaccine stability, delivery, and immunogenicity (FIG. 1). The LION-repRNA-CoV2S construct elicits robust anti-SARS-CoV-2 spike protein IgG antibody isotypes, indicative of a Type 1 T-helper response, in mice. Importantly, a single-dose administration in NHP elicited antibody responses that potently neutralized SARS-CoV-2.

LION nanoparticles represent a stable and easily manufacturable strategy for malaria vaccines: The vaccines herein do not need to be encapsulated into lipid nanoparticles under a regulated manufacturing process; formulating variant-specific vaccines with LION is more flexible and can be rapidly customized. The lipid component can be produced and stockpiled separately and then combined with the target-specific RNA as desired. The LION formulation can be stored long-term at 4° C., rendering it practical for use in low-and-middle income countries (LMICs).

LION nanoparticles are highly immunogenic in malaria models: Applicant has employed the nanostructured LION particle to deliver repRNA to prime immune responses to the P. yoelii CS antigen (repRNA-PyCS) (FIG. 2A). Vaccine immunogenicity was evaluated by serum ELISAs (FIG. 2B) and splenocyte IFNγ ELISPOTs (FIG. 2C). Anti-PyCS IgG antibody levels were substantially higher than after PyRAS immunization alone and were boosted by either a second dose of repRNA-PyCS or a dose of PyRAS (FIG. 2B). Boosting by PyRAS confirmed the fidelity of PyCS epitope presentation by repRNA-LION-expressing cells. When a single dose of repRNA-PyCS was benchmarked against gene gun PyCS (the basis for priming in the first-generation prime-and-trap vaccine), the LION repRNA-PyCS vaccine elicited >10-fold more IFNγ-producing T cells than gene gun vaccination (FIG. 2C). However, two doses of repRNA-PyCS alone do not provide sterile protection against Py wild-type SPZ challenge in mice (FIG. 2D-E). These repRNA-PyCS data support the robust and consistent immunogenicity of this platform and its ability to synergize with liver-targeted trapping vaccines to provide high levels of protection.

LION nanoparticles accelerate the protective prime-and-trap vaccination schedule: Applicant next attempted prime-and-trap vaccination, using the SU repRNA-PyCS to prime and WO PyRAS to trap. Applicant tested the prime-and-trap approach for immunogenicity and efficacy with several control groups, including two doses of repRNA-PyCS, trap-only (having an irrelevant repRNA-PfCS control followed by PyRAS), a single dose of PyRAS alone, and naïve animals (FIG. 3A). Applicant compared different prime-and-trap regimens on accelerated schedules (1-day and 5-day) vs. the standard 28-day schedule. None of the mice that received two doses of repRNA-PyCS were protected, while mice treated with PyRAS (a “trap-only” vaccine) were only 40% protected (FIG. 3B-C). However, all immunization schedules of prime-and-trap regimen produced sterile (100%) protection against wild-type PySPZ challenge in mice (FIG. 3B-C).

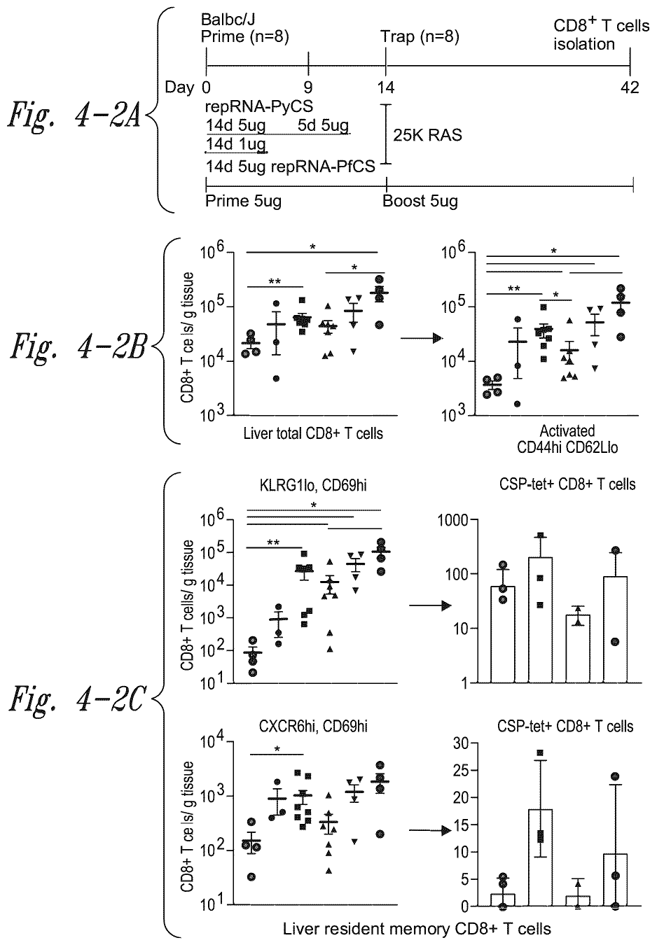

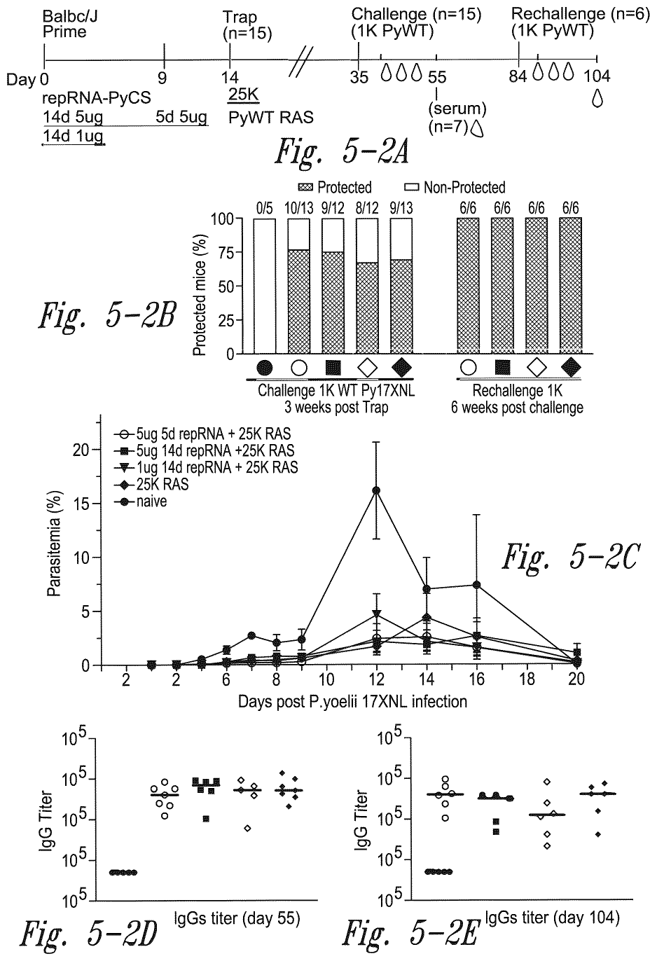

LION nanoparticles can induce a broad antibody response against multiple antigens: Using a three-antigen mixture delivered by repRNA vaccination (PyCS, PfCS, and PvCS), Applicant achieved broad humoral immune responses (FIG. 4A). The magnitude of each response from the mixed vaccine was slightly lower than the response from the single antigens alone (FIG. 4B), but consistently positive, ruling out the possibility of immune interference. Prior work has shown that adding non-CSP antibodies can rescue suboptimal CSP titers, further mitigating concerns of immune interference. Indeed, synergy was observed between antibodies targeting multiple Plasmodium antigens using passive transfer of antibodies against CS and UIS3 (up-regulated in infective sporozoites gene 3), both of which were completely non-protective on their own but elicited 64% sterile protection in combination in mice challenged with SPZ (FIG. 5). This agrees with further prior work that showed suboptimal anti-CS antibodies can be augmented by anti-SSP2 (sporozoite surface protein 2) antibodies. These data indicate that repRNA priming can provide the foundation for reliable, multi-antigen vaccination as a path to more robust and long-lived sterile protection.

The data above indicate as follows:

Accelerated Prime-Trap immunization against liver-stage malaria can be optimized. For example, following priming with repRNA-PyCS, mice or other patients including humans will be boosted with a trapping dose of PyRAS at various intervals and challenged one month after trapping. Other groups will be subjected to immunogenicity endpoints using ELISA, ELISpot, and/or flow cytometry to assess the mechanisms of protection. The results indicate that the efficacy of the best schedule of immunization for our prime-and-trap strategy will be improved. The anti-PyCS and control anti-PfCS antibody responses elicited by the immunization regimens herein can be optimized. Cellular immune responses to immunization based on the regimens herein can be optimized.

Turning to some further examples, similar to some of the comments above, while mRNA has a short half-life and period of antigen production, self-amplifying (replicon) RNA (repRNA) as formulated herein (FIG. 1-1) initiates biosynthesis of antigen-encoding mRNA in the host, raising and prolonging antigen expression and enhancing humoral and cellular immune responses. Moreover, repRNA elicits more robust immune responses after a single dose than conventional mRNA formulations, offering an attractive approach for emerging infectious diseases, such as Dengue, Zikaand SARS-CoV-2. In some embodiments herein, the use of LION eases manufacturing of malaria vaccines.

The LION oil-in-water emulsion nanocarrier was used to deliver repRNA to prime immune responses to the P. yoelii (Py) CS antigen (repRNA-PyCS) (FIG. 1, 2-1A). Immunogenicity was evaluated by serum ELISAs (FIG. 2-1B) and splenocyte IFNγ ELISPOTs (FIG. 2-1C). Anti-PyCS IgG antibody levels were substantially higher than after PyRAS immunization alone and were boosted by either a second dose of repRNA-PyCS or a trap dose of PyRAS (FIG. 2-1B). Boosting by PyRAS confirmed the fidelity of PyCS epitope presentation by repRNA-LION-expressing cells. When a single dose of repRNA-PyCS was benchmarked against a prime dose via gene-gun PyCS, the LION repRNA-PyCS vaccine elicited >10-fold more IFNγ-producing T cells than gene-gun priming (FIG. 2-1C). However, two doses of repRNA-PyCS alone (prime-boost) did not induce sterile protection against Py WT SPZ challenge (FIG. 2-1D), so Applicant developed approaches for inducing both antibodies and T cells. Nevertheless, these repRNA-PyCS data indicate the robust and consistent immunogenicity of Applicant's platform and its ability to synergize with liver-targeted trapping vaccines to provide high levels of protection.

LION nanocarriers accelerated the protective prime-and-trap (P&T) vaccination schedule. Applicant compared different P&T regimens on an accelerated schedule (5-day) vs. the standard 28-day schedule (FIG. 3-1A). Consistent with findings described above, none of the mice that received two doses of control repRNA-PyCS were protected, and mice treated with PyRAS (a “trap-only” vaccine) were only 40% protected (FIG. 3-1B). Yet, all immunization schedules of the P&T regimen produced sterile (100%) protection against WT PySPZ challenge in mice (FIG. 3-1B).

LION nanocarriers induced a broad antibody response against two antigens. By immunizing mice with a two-antigen mixture (repRNA-PyCS and-PfCS) delivered in separate LION formulations, Applicant achieved broad humoral immune responses (FIG. 4-1A, 4-1B) with a comparable magnitude of response from the mixed vaccine and single antigens alone (FIG. 4-1B), indicating that under these conditions, any immune interference present is insignificant. These data support the use of a mixture of multi-antigen encoding repRNA as prime dose leading to more robust and long-lived sterile protection.

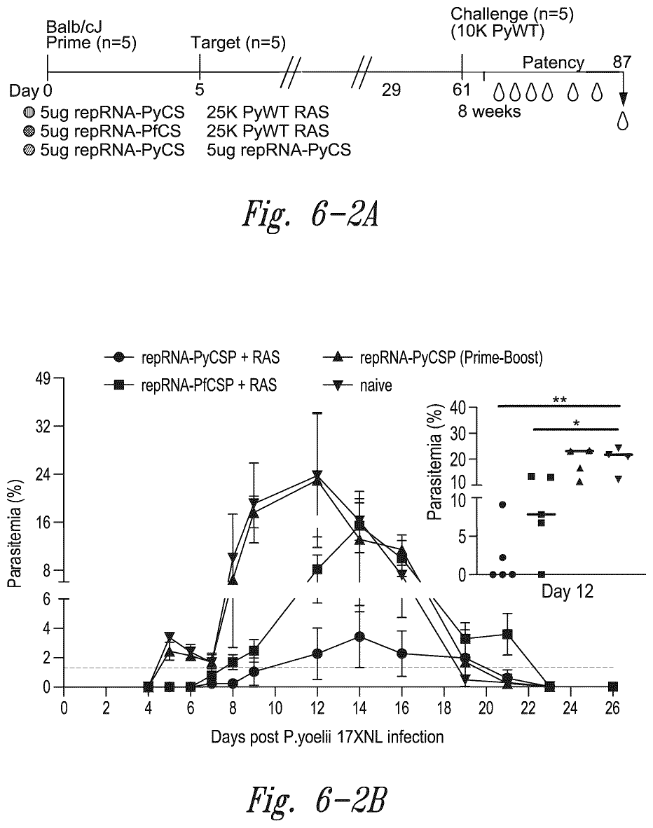

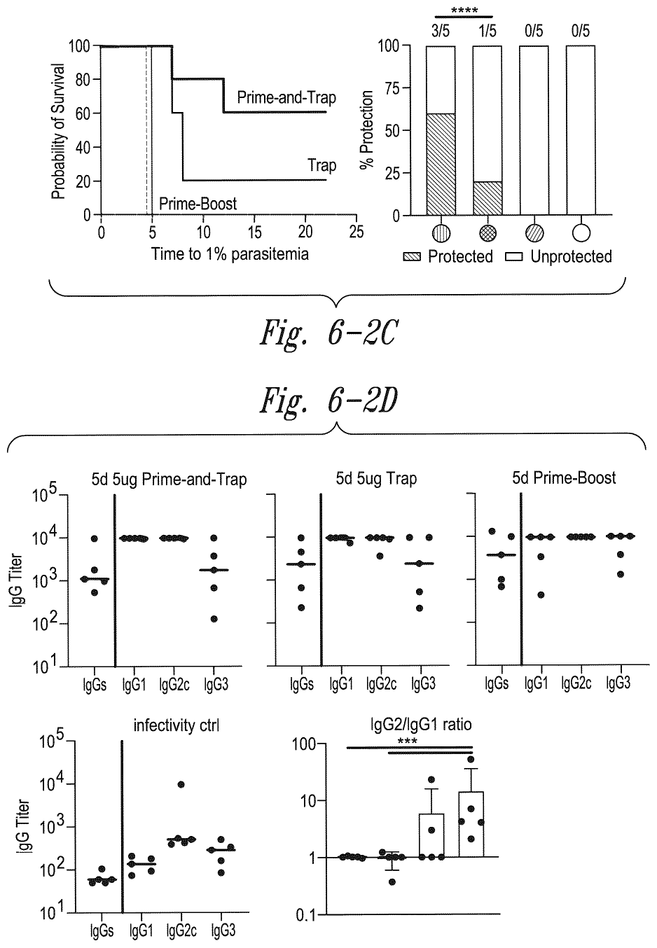

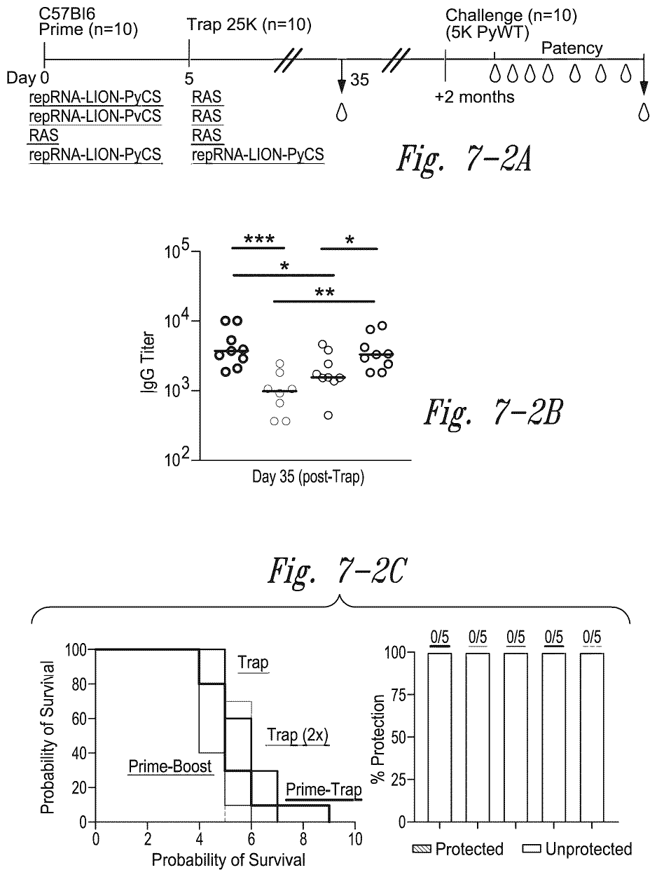

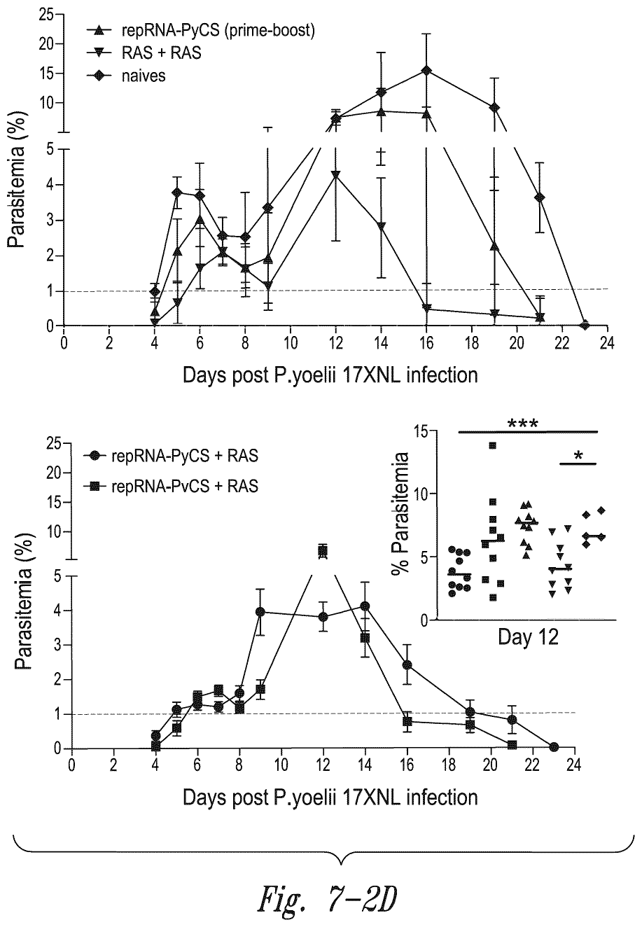

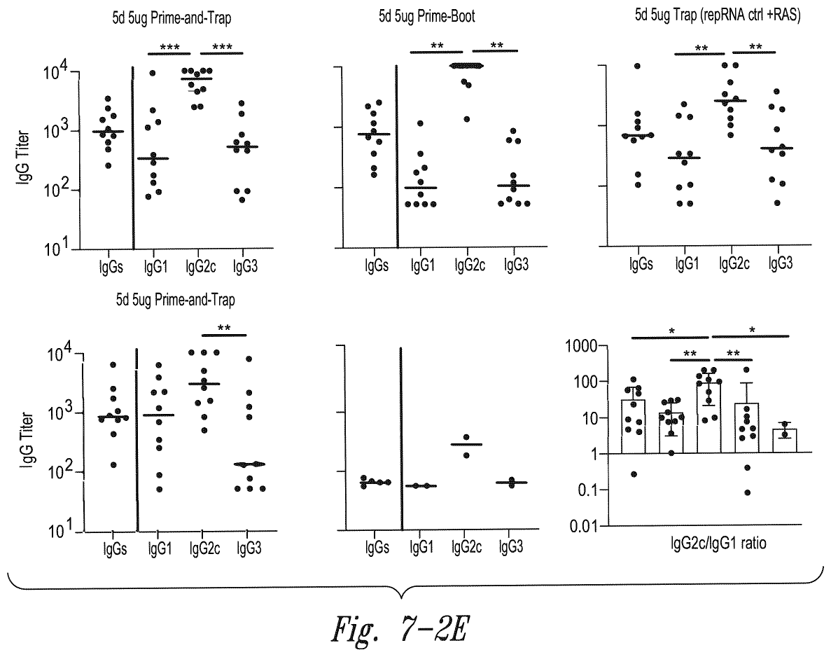

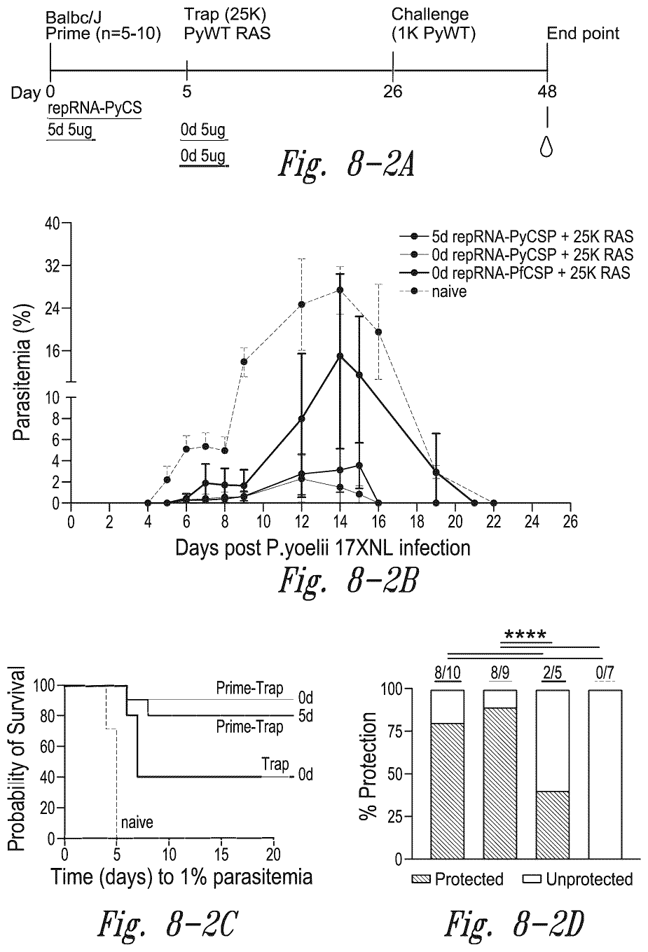

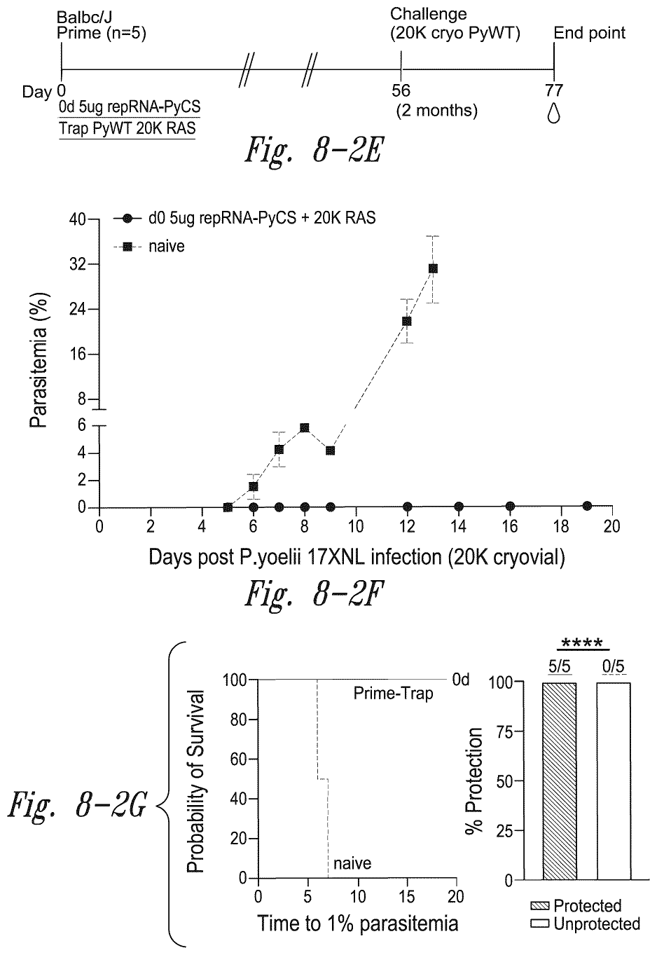

Sterile protection following same-day prime-and-trap vaccination schedule. Applicant's data shows that a 5-day P&T vaccination conferred protection in mice when challenged two months post-trapping (FIG. 6-1A). The 5-day regimen induced numbers of CS-specific liver-resident memory CD8⁺T cells (Trm) similar to the trap-only group (FIG. 6-1B) indicating long-term protection. Indeed, 60% (3/5). Female mice were protected by repRNA-PyCS P&T, compared to none (0%, 0/5) in the control repRNA-PyCS mice and only 20% (1/5) protected in the trap-only (RAS) group (FIG. 6-1C). The same-day P&T regimen conferred 100% sterile protection in all female mice at two months (5/5, FIG. 6-1C).

### FURTHER EXAMPLES

### RepRNA-CS Vaccine Formulation and Prime-Boost Immunogenicity in Balb/cJ Mice

Using the attenuated Venezuelan equine encephalitis (VEE) virus TC-83 strain Applicants incorporated the coding sequences of the CS full length protein from Plasmodium yoelii into the alphavirus expression vector to create a repRNA malaria vaccine. The coding sequences of the CS full length protein from P. falciparum and P. vivax were incorporated into the same expression vector as controls (Supplemental FIG. 1-2A). After RNA transcription and capping, the repRNA-PyCS or -PvCS or -PfCS were verified by denaturing gel electrophoresis (Supplemental FIG. 1-2B) and then transfected into mammalian cells for validation. Western blot analysis showed the expression of CS proteins to be higher than the expected molecular weight (MW) (observed vs expected: PyCS ˜99Kd vs 44.7Kd, PfCS ˜70Kd vs 43.4Kd, PvCS ˜60Kd vs 36.9Kd, respectively), probably due to the presence of surrounding glycosylation(s) that have not been totally cleaved, impairing the protein migration into the gel (FIG. 1-2A, Supplemental FIG. 1-2C).

The repRNA-CS vaccines were formulated with a LION oil-in-water emulsion (FIG. 1B). Unlike current mRNA vaccines, the LION/repRNA vaccine platform utilizes an ad-mixture formulation of LION, a highly stable cationic (DOTAP) squalene emulsion embedded in a hydrophobic oil phase (as in FIG. 1-2B) that can be manufactured independently of the RNA component and combined by a simple mixing step, e.g., at 1:1 (v/v), with the repRNA-construct for example 30 minutes prior to immunization.

To determine the immunogenicity of homologous prime-boost LION/repRNA-CS vaccination for single or dual CS antigens, mice were immunized with an IM prime of 5 μg of LION/repRNA followed by a homologous boost 14 days later (Supplemental FIG. 2-2A). The mice either received LION/repRNA vaccines for each antigen (PyCS or PfCS; 5 mg per antigen) or the combination of two antigens (PyCS and PfCS, or PfCS and repRNA-GFP (green-fluorescent protein) control; 2.5 mg per antigen). A repRNA-GFP construct was used here as a non-malaria coding antigen control. Final bleeds were collected three weeks post-boost and immune responses were analyzed by ELISA against their corresponding CS tandem repeat region. The humoral immune responses were detected against the corresponding CS antigen with little to no cross-reactivity against the heterologous CS (Figure Supplemental 2-2B). Post-immunization, all mice other than naïve mice (which were only challenged) seroconverted with a magnitude of CS-specific responses from the two-antigen based vaccines slightly lower than from the single CS antigen vaccine (p=0.0286 for PyCS compared to PyCS+PfCS; p=0.7302 for PfCS compared to PyCS+PfCS; Figure Supplemental 2-2B), which could be due to the reduced dose per antigen in the former. Nevertheless, both 5 mg or 2.5 mg dose of a prime-boost vaccine are highly immunogenic. These results demonstrate that the LION/repRNA-CS platform can induce antibody responses against malaria antigens alone or in combination, and indicate that strong immune interference does not occur when two antigens are mixed into the vaccine.

### Immunogenicity and Efficacy of a Two-Dose Prime-Boost RepRNA-PYCSP Vaccine in Balb/cJ Mice

To confirm antibody responses to homologous prime-boost LION/repRNA-PyCS vaccination in cohorts of Balb/cJ, 15 mice were immunized with an IM injection 14 days apart (FIG. 2-2A). The LION/repRNA-PfCS or LION/repRNA-PvCS vaccines were used as Applicant's control group in a cohort of 7 mice each. Any LION/repRNA-CS formulated vaccine tested was safe and well-tolerated as all mice vaccinated in any cohort exhibited normal behavior and health through the immunization study indicating no adverse health effect due to the vaccination (Supplemental FIG. 3-2A). Antibody responses were evaluated by ELISA following the prime (day 13) and boost (day 29) against peptides containing their corresponding CS tandem repeat sequence (FIG. 2-2B). The anti-PyCS total IgGs antibody levels in primed mice (D13, p=0.001, FIG. 2-2B) were substantially higher than naïve mice, and were enhanced by a second dose of LION/repRNA-PyCS (D29, p=0.028, FIG. 2-2B) with slight cross-reactivity observed against the PfCS epitope (FIG. 2-2B).

To assess efficacy, 13/15 mice from the LION/repRNA-PyCS cohort, 5/7 from the LION/repRNA-PfCS cohort and 7/7 from LION/repRNA-PvCS cohort were then challenged 3 weeks post-boost with an intravenous injection of 1000 wild-type (WT) Py 17XNL spz freshly dissected from mosquito salivary glands. This prime-boost of repRNA-PyCS vaccination alone did not provide sterile protection (0% protection) against Py wild-type spz challenge in mice (FIG. 2-2C), However, ELISA at the endpoint (post challenge (noted as “term”, at day 48) showed that the antibody levels were recalled (10 times higher) by the challenge dose of WT spz (p<0.0001 relative to naives, FIG. 2-2B. These repRNA-PyCS data support the robustness and consistency of the antibody responses induced by this repRNA platform, however this homologous vaccine strategy was not sufficient for providing sterile protection against intravenous Py malaria infection.

### Superior T Cell Immunogenicity of RepRNA-PYCS Over Gene Gun DNA Priming In Balbc/J Mice

To assess T cell responses to LION/repRNA-PyCS, and to evaluate if LION/repRNA-PyCS might be suitable to use as a priming dose in prime-and-trap vaccination, additional cohorts of Balbc/J mice were immunized with a single IM injection and responses were evaluated by splenocyte IFNγ ELISPOTs four weeks later (FIG. 2-2D). Responses were compared to splenocytes from mice immunized with a single repRNA-PfCS or gene gun administered DNA encoding PyCS (gg DNA-PyCS), which has been used as the priming dose in the first-generation prime-and-trap vaccine using RAS. The naïve mice and the mice primed with the control LION/repRNA-PfCS did not recognize the PyCS epitope by ELISPOT. In comparison, the LION/repRNA-PyCS vaccine elicited a response that was more than 10-fold increase in IFNγ-producing T cells compared to gene gun DNA-PyCS vaccination (p=0.0085, FIG. 2-2D), indicating robust priming of CD8+ T cells following a single LION/repRNA-PyCS injection.

### Humoral Responses Following Prime-and-Trap RepRNA-PYCS-RAS Vaccination in Balbc/J Mice

To assess LION/repRNA-PyCS as the priming dose in prime-and-trap vaccination, cohorts of Balbc/J mice were immunized with LION/repRNA-PyCS followed by PyRAS, along with control cohorts including two doses of homologous prime-boost repRNA-PyCS, or PyRAS trap-only (consisting either of an irrelevant repRNA-PfCS control followed by PyRAS or a single dose of PyRAS alone), and naïve animals were used as additional controls. Previously established prime-and-trap vaccines studies have a four week (28-day) schedule between doses and utilized 20,000 to 50,000 for a RAS trapping dose. To investigate if the trap dose of 25,000 PyRAS and an accelerated schedule could be achievable with the repRNA LION formulated vaccine, mice were primed IM with repRNA-PyCS (1 mg or 5 mg) 14 or 5 days prior to a PyRAS trapping dose (FIG. 3-2A). All mice immunized with this heterologous vaccination as shown by the 14-day vaccine regimen using 5 mg of repRNA-PyCS as prime dose and 25,000 RAS trapping dose remained healthy throughout the vaccination study (Supplemental FIG. 3-2B).

To assess their CS-specific whole IgG level and parasite burden post trapping, Applicants collected sera, spleens, and livers from seven mice from each cohort within 5-6 hours of the trapping dose. Mice immunized with both 14-day regimens (1 mg or 5 mg) have higher antibody titers than mice immunized with a 5-day regimen of repRNA-PyCS and the irrelevant repRNA-PfCS regimen with RAS cohort (Trap alone, FIG. 3-2B). No difference was observed between cohorts regarding the parasite burden in the spleen, while the RAS cohort (Trap alone) and the 5-day cohort are the two cohorts that had higher parasite burden in the liver (FIG. 3-2B). These results indicate that the specific anti-PyCS IgGs generated during the 14 days following the prime dose, but not within 5 days, were targeting the RAS after the trap dose was injected, reducing their distribution to the liver target.

Next, to determine if a balanced or skewed IgG subclass response was induced by repRNA-PyCS, in a separate cohort of mice Applicants measured the circulating IgG subclasses four weeks post-trapping dose using CS peptide ELISA (FIG. 3-2C). There was no significant difference between the IgG1, IgG2a and IgG3 levels or the IgG2a/IgG1 ratio between any cohort immunized with repRNA-PyCS as the priming dose, indicating that the two-dose regimen induces a balanced Th1/Th2 antibody response. However, the cohort receiving the trapping dose RAS alone (i.e., 14-day repRNA-PfCS and RAS, FIG. 3-2C, dark blue data) was the only cohort of mice with skewed Th2 type humoral immune response. Applicant's results highlight the implication of IgG2a in addition to IgG1 and IgG3 subclasses shifting the exclusive IgG1-biased humoral immune response observed after a WO-based vaccine like RAS, to a balanced Th2/Th1 type response.

### CD8⁺T Cell Responses Following Prime-and-Trap RepRNA-PYCS-RAS Vaccination In Balb/cJ Mice

To assess CD8⁺T cells responses to LION/repRNA-PyCS prime-and-trap vaccination, mice were immunized with the different immunization schedules (5 mg as priming dose in a prime-and-trap 14-day vs 5-day regimen; 5 mg prime-boost or 25,000 spz trap alone regimen) as described above (FIG. 4-2A). Livers were collected 28 days after the last immunization from two independent experiments, and lymphocytes were isolated, purified, and stained for flow cytometry as described previously. Total CD8⁺T cells and activated CD8⁺T cells (CD44^{hi} CD62L^{lo}) in the liver of prime-and-trap (5 mg 5-day, 5 14-day, 1 mg 14-day) or trap only (i.e., control prime-and-trap (5 mg 14-day ) or 25,000 RAS) immunized mice were significantly higher than the homologous prime-boost repRNA-PyCS immunized mice (FIG. 4B). To determine if Applicant's prime-and-trap vaccine can generate CS-specific liver resident memory CD8⁺T cells, CS tetramer labelled CD8⁺T cells were identified by either CD69⁺/KLRG1lo or CD69⁺/CXCR6⁺ expression. Both populations of CS-specific liver resident memory CD8⁺T cells were higher in the 5-day prime-and-trap regimen or RAS immunized mice compared to the 14-day prime-and-trap immunized mice (FIG. 4-2C), consistent with liver burden post-trapping being lower in the 14-day group as shown above.

### Efficacy of Accelerated Prime-and-Trap RepRNA-PYCS-RAS Vaccination in Balb/cJ Mice

To evaluate if the immune responses induced by prime-and-trap immunization could provide sterile immunity, cohorts of mice immunized with a LION/repRNA-PyCS as prime dose followed by the RAS for the trapping dose (as described above in FIG. 3-2) were challenged three weeks later with a dose of 1,000 freshly dissected WT Py spz delivered intravenously (FIG. 5-2A). Upon challenge, naïve mice were not protected whereas 66% of trap only and 80% of prime-and-trap mice were sterilely protected from (FIG. 5B), which is statistically equivalent. However, for the mice with breakthrough parasitemia in those immunized cohorts, Applicants saw a consistent 2 to 3-day delay in the onset of blood-stage with a reduced peak of the parasitemia (FIG. 5-2C). Two weeks post-challenge, sera was collected, and total IgG levels were quantified by ELISA. The total IgG titer post-challenge was similar in all immunized cohorts (prime-and-trap vs trap alone) indicating a recall of the CS-specific humoral immune response following sporozoite challenge (FIG. 5-2D).

The four cohorts with partial sterile protection post-challenge were then re-challenged 6 weeks later and were all protected (FIG. 5-2B) with a high level of circulating specific anti-CS IgGs (FIG. 5-2E). Altogether, Applicant's data demonstrate that the prime-and-trap strategy herein provides superior results. Applicant's prime-and-trap vaccine also improves protection against more stringent challenge in Balb/cJ mice compared to trap-alone vaccine.

To determine if the prime-and-trap vaccination can protect against a more stringent challenge, efficacy of a 2-dose 5-day prime-and-trap immunization was compared to the efficacy of a single trap-immunized mice (irrelevant prime repRNAPfCS+RAS) against a challenge with 10K Py WT spz at 8 weeks post trap (FIGS. 6-2A to 6-2B). For this, Balb/cJ mice were vaccinated with 5 mg rep-RNA-PyCS or the irrelevant rep-RNA-PfCS once followed by a 25,000 dose of RAS 5 days later. As control cohort, mice were immunized 5 days apart with 2 injections of 5 mg rep-RNA-PyCS as the homologous prime-boost cohort. Even though this experiment was not replicated independently, Applicants observed similar results than previously reported, i.e., the homologous prime-boost repRNA-PyCS cohort showed no protection following challenge (FIGS. 6-2B, 6-2C, orange data), and was indistinguishable from Applicant's control infectivity cohort (FIGS. 6-2B, 6-2C, black data). Compared to Applicant's 3-week challenge cohort where the trap only (RAS) vaccinated mice immunized in a 5-day interval yielded a 66% efficacy against challenge (FIG. 5-2D), in this 8-week challenge cohort with a higher challenge dose, the efficacy was reduced to 20% (FIG. 6-2C, blue data). However, the 5-day regimen prime-and-trap yielded a significantly higher level of sterile protection than trap-alone immunization with 70% efficacy against challenge (FIG. 6-2C, green data). Regarding the humoral immunity post-challenge, high titer of anti-CS IgGs were detected for all prime-and-trap and no difference was observed in IgG subclass between the three repRNA immunized cohorts (FIG. 6-2D) when post-challenge sera was tested by ELISA.

### Prime-and-Trap RepRNA-PYCS-RAS Vaccination Does Not Elicit Sterile Protection in C57BL6 Mice

To further investigate the requirement of the CS-specific CD8⁺T cells epitope (SYIPSAEKI) as necessary for protection, C57BL/6 mice that are unable to present this specific epitope were immunized and challenged. C57BL/6 mice have an H-2-K^{d} restricted epitope and do not express the relevant MHC-I allele to present the SYIPSAEKI epitope in the infected hepatocytes. Mice were vaccinated in a 5-day regimen with 5 mg repRNA-PyCS and 25,000 RAS (FIG. 7-2A). The prime-and-trap cohort (repRNA-PyCS+RAS) were compared to a prime-boost repRNA-PyCS cohort, a trap cohort (repRNA-PvCS+RAS), a double trap (RAS+RAS) cohort and infectivity control cohort. One month (at day 35) post second immunization, sera was collected and analyzed by ELISA against the PyCS peptide (FIG. 7-2B). Both trap and prime-boost cohorts had significantly lower IgG titers compared to the double RAS or the prime-and-trap cohorts, which both have equivalent and strong anti-CS antibody titers (FIG. 7-2B).

Within this experiment, animals were then challenged 8 weeks later by the intravenous dose of 5,000 infectious WT P. yoelii spz. As anticipated, none of the C57BL/6 mice cohort had sterile protective immunity following challenge (FIG. 7-2C) but the trap, double trap and the prime-and-trap showed a delay of onset parasitemia compared to the infectivity control or prime-boost cohorts (FIG. 7-2C). Nevertheless, relative to the infectivity control cohort (FIG. 7-2D, black data), animals immunized with either prime-and-trap (FIG. 7-2D, green data) or double trap (FIG. 7-2D, blue data) showed a significantly greater control of the parasitemia (particularly at the day of the peak of infection) than the cohorts immunized with either irrelevant repRNA-PvCS and RAS (FIG. 7-2D, purple data) or prime-boost double repRNA (FIG. 7-2D, orange data). Interestingly, while the antibody response to the homologous prime-boost repRNA immunization was biased towards IgG2c (FIG. 7-2E, orange data), all the other regimens had IgG2c:IgG1 ratios that were significantly biased toward a balanced response (FIG. 7-2E, prime-and-trap in green, prime control-and-trap in purple and double trap (RAS) in blue).

### Sterile Protection Following Same-Day Prime-and-Trap RepRNA-PYCS-RAS Vaccination

As described above, accelerating the prime-and-trap vaccination from a 14-day to a 5-day immunization schedule surprisingly helped reduce the circulating CS specific antibodies (FIG. 3-2B) and improved numbers of CSP+ liver CD8⁺Trm cells (FIG. 5) while maintaining high efficacy (FIG. 4-2E). The protective efficacy was next assessed in Balb/cJ mice in a same-day schedule of immunization compared to the 5-day schedule. Mice were vaccinated in a same-day (0-day) or a 5-day regimen with 5 mg repRNA-PyCS and 25,000 RAS and challenged 3 weeks later by the intravenous dose of 1,000 freshly dissected infectious WT P. yoelii spz (FIG. 8-2A). Those cohorts were compared to a trap alone (repRNA-PfCS+25,000 RAS) cohort and the infectivity control cohort. While both the trap cohort (3/5 of the mice) and control cohort (5/5 of the mice) developed a high parasitemia quickly, both prime-and-trap cohorts (5-day vs 0-day) have only a small subset of mice that developed a parasitemia (2/10 and 1/9, respectively) hence providing 80% and about 90% sterile protection respectively (FIGS. 8-2B to 8-2D). Another important observation was that this onset of parasitemia appeared 10 days post infection and was cleared by day 16 (FIG. 8-2B) whereas in both control cohorts the parasitemia lasted for 15 days and was cleared by day 22 post-challenge. To determine if a same-day prime-and-trap vaccine can yield protection following a more stringent challenge, a cohort was immunized with 5 mg repRNA-PyCS and 25,000 RAS on the same-day and was challenged two months later with 20,000 of infectious WT P. yoelii spz that were previously frozen in-house as described and thawed and injected within 1 hour (FIG. 8E). While the infectivity control cohort rapidly developed an onset parasitemia, 100% of the mice were surprisingly sterilely protection in Applicant's vaccinated cohorts (FIGS. 8-2F to 8-2G). These data indicate that an accelerated same-day prime-and-trap immunization scheduled with 2 injections concurrently via IM and IV routes respectively can provide sterile protection to a patient mammal.

Malaria vaccine development has significant promise for life-saving benefit and to reduce the global burden of malaria. Applicant's prime-and-trap approach combines two vaccination strategies that have limited stand-alone efficacy, but together synergize to provide surprising advantages and superior results. Applicants showed that a homologous LION/repRNA-PyCS vaccine (prime-boost) is highly immunogenic, for example at the various doses evaluated, ranging from 1 mg to 5 mg eliciting strong antibody responses when given in a 2-dose immunization regimen 2-weeks apart. Antibody levels tend to be modest following the priming dose which increased following a booster dose. Applicants demonstrated that homologous LION/repRNA-CS vaccination alone was not sufficient to prevent blood stage infection and hence provide protection. The methods, compositions, systems, etc., herein overcome these issues and reduce the cost of vaccinating people in low resource environments, for example by reducing the cost of goods for the repRNA vaccine, dose sparing for the attenuated spz, and the significant advantage of a single clinic visit for vaccination against malaria. Applicant's priming dose using emulsion nanocarriers comprising replicon RNA (repRNA) operably connected to at least immunological components of an immunogenic plasmodium protein, such as a LION/repRNA-PyCS, for a heterologous prime-and-trap vaccine approach, is surprisingly highly immunogenic. For example, Applicants induced a strong humoral response in Balb/cJ and C57Bl/6 mice when given 2 weeks apart or on the same-day compared to the trapping dose alone, and observed a strain-specific CD8 T cell response in Balb/cJ mice. Additionally, Applicants found a 2-3-day delay in the blood-stage parasitemia compared to the control groups, indicating that some level of protection was conferred. To Applicant's knowledge, Applicant's prime-and-trap vaccine approach has the advantage that immunized patients developed humoral and cellular immunities without waning down the CD8⁺T cells achieved by the injection of the RAS (or otherwise attenuated WO) trapping dose, and inducing the ability to shift the antibody profile toward a balanced Th1/Th2 humoral immune response.

The Examples herein demonstrate a multi-component vaccination approach for the concurrent induction of humoral and T cell immunities using a repRNA-CS formulated with LION nanoparticle and PyRAS targeting the liver.

### FURTHER EXAMPLES

### Vaccine Design

The chosen antigen for Applicant's vaccine design is the full length circumsporozoite (CS) protein from Plasmodium, as described in detail in Supplemental FIG. 1-2. This antigen contains an immunodominant and protective CD8⁺T cell epitope specific to the H-2K^{d} (Balb/cJ)-restricted genetic background, and two distinct dominants for CD8⁺epitopes in the H2K^{b}/^{b} (C57BL/6)-restricted genetic background.

### RNA Production and Lion Formulation

Full-length CS coding sequences for Pf, Pv and Py was cloned separately into a Venezuelan equine encephalitis (VEE) replicon vector (pT7-VEE-Rep). In vitro transcription was performed at 34° C. using a T7 MEGAscript T7 Transcription kit (Invitrogen). RNA was purified via lithium chloride precipitation, followed by capping with a capping kit (New England Biolabs) as described. RNA was further purified and stored in −80° C. until use. Denatured repRNA were verified by electrophoresis in a 1% agarose gel. Briefly, 2 mg of each repRNA were denatured by glyoxal treatment (NothernMax-Gly, AM8551, ThermoFisher), and run in an agarose gel with NorthernMax-Gly gel prep/running buffer (AM8678, ThermoFisher). The bands were visualized by ethidium bromide premixed into the solution and analyzed by a Biorad gel docXR+Imaging system.

To protect the RNA replicons from degradation, Applicants combined each one with LION nanoparticles obtained from HDT Biosuch. In brief, the LION formulation has inorganic SPIO nanoparticles within a hydrophobic squalene core to enhance formulation stability. LION particles were manufactured by combining the iron oxide nanoparticles with the oil phase (squalene, Span 60, and DOTAP) while the aqueous phase, containing Tween 80 and sodium citrate dihydrate solution in water, was prepared separately. The oil and aqueous phases were then mixed and emulsified then processed by passaging through a microfluidizer to reach 50±5 nm with a 0.2 polydispersity index. The microfluidized LION was terminally filtered with a 200-nm pore-size polyethersulfone filter and stored at 2° to 8° C.

### Cells Lines

To qualify the vaccine candidate in vitro, BHK cells (American Type Culture Collection (ATCC)) were transfected with repRNA or mock transfected using a OptiMEM (Gibco) and Expifectamine transfection kit (ThermoFisher). Cells were scrapped off and lysed with RIPA buffer 24-48 hours later, and lysates were analyzed by SDS-polyacrylamide gel electrophoresis and by Western blot.

### Western Blots

Cells lysates were analysis by Western blot after transfer to nitrocellulose membrane. For detection, anti-rabbit polyclonal anti CSP (Py, Pf or Pv) (Pocono) were used (1/1,000) followed by goat anti-rabbit IgG (H+L) Alkaline phosphatase secondary antibody (Invitrogen, T2191) (1/10,000).

### Mice

Female Balb/cJ mice and C57Bl/6 (B6) mice, six to eight weeks old, were purchased from The Jackson Laboratories (Bar Harbor, ME, USA). Mice were maintained under pathogen-free conditions in animal facilities and were fed with autoclaved food ad libitum. Mice were housed and cared for in standard IACUC-approved animal facilities from Bloodworks Northwest and used in compliance with IACUC-approved protocols.

### Lion/repRNA Vaccination

For all LION/repRNA vaccines, five micrograms of RNA was mixed with LION and injected into the mice intramuscularly (IM) using a total of 50 μl (25 μl in each leg). A two-vial formulation method was performed as described and Applicant's immunization protocol and timeline are described in each respective figure.

### Sporozoite Isolation, Vaccination and Challenge

Wild-type Py (17XNL strain) sporozoites were prepared by cyclical transmission in Balb/cJ mice and Anopheles stephensi mosquitoes at the Seattle Children's Center for Global Infectious Disease Research Insectary (Seattle, WA, USA). Female 6- to 8-week-old SW mice were injected with blood-stage Py 17XNL WT parasites to begin the growth cycle and used to feed female Anopheles stephensi mosquitoes. At day 15 after blood meal, salivary gland sporozoites were isolated and harvested as previously described.

RAS were generated by exposure to 10,000 rads using an X-ray irradiator (Rad-Source, Suwanee, GA, USA). RAS was resuspended in 100 mL Schneider and administered to the mice through tail-vein injection.

Infectious sporozoite for challenge were prepared in an equivalent manner but without irradiation. All experimental and control mice were challenged with live Py 17XNL sporozoites.

### Liver Lymphocyte Flow Cytometry

Livers were perfused with 10 ml PBS with 2 mM EDTA by injection into the portal vein, with outlet drainage via the inferior vena cava. Gall bladder was removed, and livers were placed in 5 ml RPMI 1640 supplemented with glutamine with 5% FBS on ice to ensure cell survival. Livers were mashed through a 200-mm mesh filter (pluriSelect, San Diego, CA) with the back of a 3-ml syringe plunger. The mesh filter and plunger were washed with FBS-/glutamine supplemented RPMI 1640. Cell suspension was spun at 80 3 g for 1 min at 4° C. without braking; supernatants were collected and transferred to a clean 50-ml conical tube where they were spun at 500 3 g for 8 min at 4° C. The cell pellet was resuspended in 10 ml room temperature 35% Percoll (GE Healthcare Life Sciences) in HBSS (Life Technologies) supplemented with 100 U heparin and spun at room temperature at 900 3 g for 25 min with no brake. The final cell pellet containing intrahepatic lymphocytes was resuspended in 2 ml ammonium-chloride-potassium lysis buffer for 2-3 min, quenched with 8 ml MACS buffer (PBS, 1 mM EDTA, 0.5% FBS), and then spun at 450 3 g at 4° C. for 5 min. Final pellets were resuspended in 100 ml MACS buffer and moved to a 96-well plate for treatment with an Fc block for 30 min (anti-CD 16/32, clone 2.4G2; BD Biosciences), Ab staining for 45 min (see Ab materials listed below), fixation for 20 min (Cytofix/Cytoperm reagent; BD Biosciences), and analysis by flow cytometry on an LSR II (BD Biosciences). The following Abs were used to assess liver Trm cells: CD3e-BUV 395 (clone 145-2C11; BD Biosciences), B220-BV711 (clone RA3-6B2; Bio-Legend), CD4-Alexa Fluor 700 (clone GK1.5; BioLegend), CD8a-BV421 (clone 53-607; BD Biosciences), CD69-BV510 (clone H1.2F3; BD Biosciences), CD44-Alexa Fluor 488 (clone IM7; BioLegend), CD62LPE-Cy7 (clone MEL-14; BD Biosciences), KLRG1-PerCP-Cy5.5 (clone 2F1/KLRG1; BioLegend), CXCR6-PE (clone 221002; R&D Systems), and CSP tetramer (CSP epitope SYVPSAEQI provided by the National Institutes of Health Tetramer Core) conjugated to streptavidin-allophycocyanin (ProZyme) per standard protocols.

Cells were gated for CD8+T cells (CD3e+, B220−, CD4−), CD44hi by CD62Llo, then assessed by either KLRG1lo by CD69hi or by CXCR6hi by CD69hi. Antigen specificity was then assessed by PyCSP-tetramer (SYVPSAEQI-specific H2-Kd tetramer). Cell count per gram of tissue was calculated based on a known concentration of counting beads per samples to normalize data.

### Ex Vivo IFNγ ELISPOT

Spleens were harvested and splenocytes separated from Balb/cJ mice at 28 days post immunization. A total of 1×10E5 splenocytes were combined with SYVPSAEQI peptide (1 mg/ml final) (Genemed Synthesis) for murine IFNγ ELISPOT (eBioscience), cultured for 18 h at 37°0 C. and developed following manufacturer guidelines. The percentage of antigen-specific T cells was calculated based on the spot-forming units counted in each well divided by the total number of splenocytes applied to each well.

### Blood Stage and Liver Burden

Breakthrough to blood stage patency was assessed by Giemsa-stained thin blood smear starting at day 4 after challenge and ending at day 21, at which time a negative smear was attributed to complete protection. Liver burden was detected by qRT-PCR from harvested liver mice 44 hr post-challenge.

Mice immunized with P. falciparum- or P. vivax-repRNA-CS and challenged with live P. yoelii spz were used as controls. Sterile protection was deﬁned as being blood smear negative, and the Kaplan-Meier curves illustrate the time to 1% parasitemia during days 4-21 after challenge with 17XNL strain P. yoelii live spz.

### qRT-PCR

Total RNA was extracted from Py infected livers using TRIzol reagent (Thermo Fisher Scientific) and treated with Turbo DNase (Ambion). cDNA synthesis was performed using a SuperScript III Platinum two-step qRT-PCR kit (Thermo Fisher Scientific). The primers used for amplification of 18S rRNA from cDNA were 18S-fwd: (GGGGATTGGTTTTGACGTTTTTGCG) and 18S-rev: (AAGCATTAAATAAAGCGAATACATCCTTAT). Mouse GAPDH was amplified with cDNA using gap dh-fwd: (CCTCAACTACATGG TTTACAT) and gap dh-rev: (GCTCCTGGAAGATGGTGATG) primers. All qRT-PCR amplification cycles was performed at 95° C. for 30 s for DNA denaturation and at 60° C. for 4 min for primer annealing and extension.

### ELISA

MaxiSorp plates were coated with 100 mL CSP (Py or Pf or Pv) peptide at 1 mg/ml in PBS. Plates were then washed with PBS+0.05% tween20 (PBS-T) and blocked with 1% BSA in PBS-T for 2 hrs at RT. Mice sera samples were plated at a dilution of 1:50 in PBS-T+0.1% BSA, serially titrated 1:3 for 6 wells, and then incubated for 2 hrs at RT or overnight at 4° C. Following washing steps, plates were incubated with secondary antibodies diluted 1:5000 in PBST+0.1% BSA for 1 hr RT. Following washing, 100 mL TMB was added per well and incubated 5-10 minutes before stopping with 50 mL sulfuric acid.

### Statistics

Comparisons of ELISA groups or flow cytometry cell counts were done using the non-parametric two-tailed Mann-Whitney U test (*p=0.05, **p=0.01, ***p=0.001, ****p<0.0001). ELISPOT assay comparisons were done by unpaired, two-tailed Student's t tests. Statistical significance between groups of mice for their spleen or liver burden qRT-PCR was evaluated using One-way ANOVA followed by Kruskal-Wallis test and Dunn's multiple comparisons test (*p<0.05, **p<0.005). Protection data was evaluated using Fisher's exact test. All groups were compared against the prime-boost cohort or the trap-alone cohort (repRNA-PfCS or repRNA-PvCS for priming, and RAS for trapping dose; ****p<0.0001). Error bars indicated are SEM of the mean with individual mouse samples shown. Statistical significance was defined as p<0.05 using Prism Graph-Pad 9.4.1 Software (San Diego, CA).

### References

1. Olsen T M, Stone B C, Chuenchob V, Murphy S C. Prime-and-Trap Malaria Vaccination To Generate Protective CD8+ Liver-Resident Memory T Cells. J Immunol Baltim Md 1950. 2018 Oct. 1; 201(7):1984-1993. PMID: 30127085
2. Watson F, Shears M, Matsubara J, Kalata A, Seilie A, Talavera I C, Olsen T, Tsuji M, Chakravarty S, Sim B K L, Hoffman S, Murphy S. Cryopreserved Sporozoites with and without the Glycolipid Adjuvant 7DW8-5 Protect in Prime-and-Trap Malaria Vaccination. Am J Trop Med Hyg. 2022 Feb. 28; tpmd211084. PMID: 35226868
3. Erasmus J H, Khandhar A P, Guderian J, Granger B, Archer J, Archer M, Gage E, Fuerte-Stone J, Larson E, Lin S, Kramer R, Coler R N, Fox C B, Stinchcomb D T, Reed S G, Van Hoeven N. A Nanostructured Lipid Carrier for Delivery of a Replicating Viral RNA Provides Single, Low-Dose Protection against Zika. Mol Ther J Am Soc Gene Ther. 2018 03; 26(10):2507-2522. PMCID: PMC6171036
4. Erasmus J H, Khandhar A P, O'Connor M A, Walls A C, Hemann E A, Murapa P, Archer J, Leventhal S, Fuller J T, Lewis T B, Draves K E, Randall S, Guerriero K A, Duthie M S, Carter D, Reed S G, Hawman D W, Feldmann H, Gale M, Veesler D, Berglund P, Fuller D H. An Alphavirus-derived replicon RNA vaccine induces SARS-CoV-2 neutralizing antibody and T cell responses in mice and nonhuman primates. Sci Transl Med. 2020 05; 12(555). PMCID: PMC7402629
5. Zhang M, Sun J, Li M, Jin X. Modified mRNA-LNP Vaccines Confer Protection against Experimental DENV-2 Infection in Mice. Mol Ther Methods Clin Dev. 2020 Sep. 11; 18:702-712.
6. Ljungberg K, Liljeström P. Self-replicating alphavirus RNA vaccines. Expert Rev Vaccines. 2015 February; 14(2):177-194. PMID: 25269775
7. Kublin J G, Mikolajczak S A, Sack B K, Fishbaugher M E, Seilie A, Shelton L, VonGoedert T, Firat M, Magee S, Fritzen E, Betz W, Kain H S, Dankwa D A, Steel R W J, Vaughan A M, Noah Sather D, Murphy S C, Kappe S H I. Complete attenuation of genetically engineered Plasmodium falciparum sporozoites in human subjects. Sci Transl Med. 2017 Jan. 4; 9(371):eaad9099. PMID: 28053159
8. Kennedy M, Fishbaugher M E, Vaughan A M, Patrapuvich R, Boonhok R, Yimamnuaychok N, Rezakhani N, Metzger P, Ponpuak M, Sattabongkot J, Kappe S H, Hume J C C, Lindner S E. A rapid and scalable density gradient purification method for Plasmodium sporozoites. Malar J. 2012 Dec. 17; 11:421. PMCID: PMC3543293
9. Tarun A S, Dumpit R F, Camargo N, Labaied M, Liu P, Takagi A, Wang R, Kappe S H I. Protracted sterile protection with Plasmodium yoelii pre-erythrocytic genetically attenuated parasite malaria vaccines is independent of significant liver-stage persistence and is mediated by CD8+T cells. J Infect Dis. 2007 Aug. 15; 196(4):608-616. PMID: 17624848
10. Minkah N K, Wilder B K, Sheikh A A, Martinson T, Wegmair L, Vaughan A M, Kappe S H I. Innate immunity limits protective adaptive immune responses against pre-erythrocytic malaria parasites. Nat Commun. Nature Publishing Group; 2019 Sep. 2; 10(1):3950.

What is claimed is:

1. A method of vaccinating a patient against malaria, comprising:
a) administering at least one priming dose that consists of a nanoparticle carrier in combination with nucleic acids encoding one or more antigenic Plasmodia proteins or protein fragments; and then
b) administering at least one trapping dose that consists of one or more antigenic Plasmodia proteins or protein fragments.

2. The method of claim 1 wherein the nanoparticle carriers are lipid inorganic nanoparticles (LIONs).

3. The method of claim 1 wherein the nanoparticle carriers are solid nanoparticles.

4. The method of claim 1 wherein the nanoparticle carriers are semi-solid nanoparticles.

5. The method of any one of claims 1 to 4 wherein said Plasmodia are taken from a group consisting of the human and zoonotic pathogens Plasmodium falciparum, Plasmodium vivax, Plasmodium malariae, Plasmodium ovale, and Plasmodium knowlesi.

6. The method of any one of claims 1 to 5 wherein the nucleic acid is a replicating RNA operably contained within a viral vector.

7. The method of claim 6 wherein the viral vector is a Venezuelan Equine Encephalitis Virus (VEEV).

8. The method of claim 6 wherein the viral vector is an adenovirus.

9. The method of any one of claims 1 to 8 wherein the at least one trapping dose comprises attenuated sporozoites from a group consisting essentially of RAS, GAP, Pb and CVac.

10. The method of any one of claims 1 to 9 wherein the at least one trapping dose comprises a replicating nucleic acid encoding one or more antigenic Plasmodia proteins or protein fragments that generates a T-cell response to the one or more antigenic Plasmodia proteins or protein fragments therein.

11. The method of claim 10 wherein the T-cell response includes liver-resident T cells.

12. The method of any one of claims 1 to 11 wherein the priming dose is administered before and on the same day as the trapping dose.

13. The method of any one of claims 1 to 12 wherein the priming dose is administered first followed by the trapping dose(s) from 12-120 hours later.

14. The method of any one of claims 1 to 13 wherein the priming dose is administered first followed by the trapping dose(s) from 121 hours to 28 days later.

15. The method of any one of claims 1 to 14 wherein the priming dose is administered first followed by the trapping dose(s) more than 28 days later,

16. An anti-malarial vaccine comprising lipid inorganic nanoparticle nanocarriers coupled to replicon RNA (repRNA) operably connected to at least one immunological component of a plasmodium CS protein.

17. An anti-malarial vaccine comprising oil-in-water emulsion nanocarriers comprising repliconRNA (repRNA) operably connected to a substantially full-length plasmodium CS protein.

18. The system of any one of claims 16 or 17 wherein the anti-malarial vaccine is a part of an anti-malarial vaccine system, the system further comprises a trapping component comprising attenuated whole organism Plasmodium sporozoites.

19. The system of claim 18 wherein the attenuated whole organism Plasmodium sporozoites are radiation attenuated sporozoites (WO RAS).

20. The system of claim 18 wherein the attenuated whole organism Plasmodium sporozoites are genetically attenuated sporozoites.

21. A method of inducing an anti-malarial immunological response in a mammal, the method comprising:
a) administering to the mammal the anti-malarial vaccine of any one of claims 16 to 20;
b) then administering to the mammal the trapping component of any one of claims 18 to 20; and,
c) thereby inducing the anti-malarial immunological response in the mammal.

22. The method of claim 21 further comprising administering the trapping component within 14 days or less of administering the anti-malarial vaccine.

23. The method of any one of claims 21 to 22 wherein the method further comprises administering the trapping component within 5 days or less of administering the anti-malarial vaccine.

24. The method of any one of claims 21 to 22 wherein the method further comprises administering the trapping component within 1 day of administering the anti-malarial vaccine.

25. The method of any one of claims 21 to 22 wherein the method further comprises administering the trapping component on the same day of administering the anti-malarial vaccine.

26. The method of any one of claims 21 to 25 wherein the trapping component is administered intravascularly (IV) and the anti-malarial vaccine is administered intramuscularly (IM).

27. The method of any one of claims 21 to 26 wherein A) the Lipid InOrganic Nanoparticle nanocarriers or the oil-in-water emulsion nanocarriers are mixed with B) the repRNA less than about 60 minutes before the administration to the mammal.

28. The method of any one of claims 21 to 26 wherein A) the Lipid InOrganic Nanoparticle nanocarriers or the oil-in-water emulsion nanocarriersare mixed with B) the repRNA less than about 30 minutes before the administration to the mammal.
